(19) United States
(12) Patent Application Publication
(10) Pub. No.: US 2008/0214951 A1
(43) Pub. Date: Sep. 4, 2008

(54) Cerebrospinal Fluid Evaluation Systems

(75) Inventors: Frederick J. Fritz, Skillman, NJ (US); Marek Swoboda, Philadelphia, PA (US)

Correspondence Address: CAESAR, RIVISE, BERNSTEIN,;COHEN & POKOTILOW, LTD., 11TH FLOOR, SEVEN PENN CENTER, 1635 MARKET STREET, PHILADELPHIA, PA, 19103-2212, US

(73) Assignee: Neuro Diagnostic Devices, Inc., Trevose, PA (US)

(21) Appl. No.: 12/055,990

(22) Filed: Mar. 26, 2008

Related U.S. Application Data
(63) Continuation-in-part of application No. 10/770,754, filed on Feb. 3, 2004.
(60) Provisional application No. 60/989,284, filed on Nov. 20, 2007.
(60) Provisional application No. 60/941,827, filed on Jun. 4, 2007.
(60) Provisional application No. 60/939,205, filed on May 21, 2007.
(60) Provisional application No. 60/911,687, filed on Apr. 13, 2007.

(51) Int. Cl. A61B 5/01; A61M 27/00

(52) U.S. Cl. 600/549; 604/8

(57) ABSTRACT

Methods and devices for testing for the presence, absence and/or rate of flow in a shunt tubing implanted under the skin by using a measurement pad having a plurality of temperature sensor configurations, or by using other temperature sensor arrangements, or by using a temperature sensitive material, which are positioned over, or in the vicinity of, the CSF shunt in substantially an upstream and downstream orientation. A temperature source, e.g., a cooling agent, is then applied at a predetermined location with respect to the measurement pad that is insulated from the temperature sensors, or to the temperature sensitive material. The movement of this temperature “pulse” is detected by the temperature sensors, or temperature sensitive material, via the shunt tubing as the CSF carries the temperature pulse while a control sensor detects the pulse via convection through the skin. The temperature data from these sensors are provided to a CSF analyzer that subtracts the control sensor data from each of the other sensors for determining a CSF shunt flow status or flow rate. A reader is used to optically or electrically detect the changes in the temperature sensitive material for determining a CSF shunt flow status or flow rate.

### CROSS-REFERENCE TO RELATED APPLICATIONS

This application is a Continuation-in-Part application of, and claims the benefit under 35 U.S.C. §120 of, application Ser. No. 10/770,754 filed on Feb. 3, 2004 entitled CEREBRAL SPINAL FLUID SHUNT EVALUATION SYSTEM, and whose entire disclosure is incorporated by reference herein. Furthermore, this utility application also claims the benefit under 35 U.S.C. §119(e) of Provisional Application Ser. Nos. 60/911,687 filed on Apr. 13, 2007, entitled CEREBROVASCULAR FLUID EVALUATION SYSTEM HAVING THERMAL FLOW AND FLOW RATE MEASUREMENT PAD; 60/939,205 filed on May 21, 2007, entitled A METHOD AND DEVICE FOR MEASURING FLOW IN TUBES IMPLANTED SUBSCUTANEOUSLY; 60/941,827 filed on Jun. 4, 2007, entitled A METHOD AND DEVICE FOR DETECTING FLOW IN SUBCUTANEOUSLY-IMPLANTED SHUNTS/TUBING USING A TEMPERATURE SOURCE DIRECTLY OVER A TEMPERATURE SENSOR; and 60/989,284 filed on Nov. 20, 2007 entitled CSF EVALUATION SYSTEM USING FAST RESPONSE TEMPERATURE SENSORS AND MEASUREMENT PAD, and all of whose entire disclosures are incorporated by reference herein.

### BACKGROUND OF THE INVENTION

1. Field of Invention

The present invention generally relates to cerebrospinal fluid shunts and, more particularly, to a method and device for testing for the presence, absence and/or rate of flow in the shunt tubing implanted under the skin.

2. Description of Related Art

A cerebrospinal fluid (CSF) shunt includes a system of tubing that allows CSF to flow from a patient's brain to another part of the body (e.g., abdomen to relieve pressure in the brain). As a result, it is desirable to know, periodically, that the pathway of the CSF shunt remains unobstructed to permit CSF flow and what the flow rate is. It is also desirable to make these determinations non-invasively when quantifying the CSF flow.

FIG. 1 depicts a prior art cerebral spinal fluid (CSF) shunt evaluation system 10. The CSF shunt evaluation system 10 includes a shunt tubing 18 that allows CSF to flow from the brain of a patient to another part of the body of the patient such as the abdomen, e.g., for treatment of a patient with hydroencephalus. The CSF shunt evaluation system 10 monitors the flow of the CSF through the shunt tubing 18 by means of upstream cooling of the CSF and a downstream sensor 14. The sensor 14 can be a temperature sensor, such as a thermistor, a thermocouple or a semiconductor sensor. The downstream sensor 14 is disposed over the shunt tubing 18 in the vicinity where the shunt tubing 18 empties into the abdominal cavity in order to detect changes in temperature as the cooled CSF is transported from the cooled region to the abdominal cavity.

The sensor 14 could be conventional temperature sensitive device wherein the internal resistance of the sensor 14 varies, either directly or inversely, according to the temperature of the sensor 14. Thus, changes in the temperature of the sensor 14 were detected by merely making a determination of its resistance or, equivalently, a measurement of the changes in the amount of current through the sensor 18.

In operation, a user of the shunt evaluation system 10 could place an ice cube on the scalp of the patient over the shunt tubing 18 for about one minute using, for example, forceps. While the safety of using ice makes it preferred for cooling the CSF, a Peltier stack maintained at zero or one degree centigrade can be used. The ice cube cooled the CSF in the shunt tubing 18 as it flowed from the scalp region toward the downstream sensor 14. The downstream sensor 14 was adapted to detect relatively small changes in skin temperature in regions over the shunt tubing 18 as the cooled CSF flowed from the head to the abdomen of the patient.

Referring now to FIG. 2, there is shown another prior art CSF shunt evaluation system 20. The CSF shunt evaluation system 20 included two sensors 24 disposed over the shunt tubing 18. The two sensors 24 were separated from each other by a known distance. The use of the two sensors 24 in the shunt evaluation system 20 in this manner permitted a determination of the flow rate of the CSF through the flow of the shunt tubing 18, in addition to a determination of whether the CSF fluid was flowing through the tubing 18. The flow rate of the CSF could then be calculated since a downward temperature deflection could be recorded for each sensor 24, and the difference in time between the deflections of the two sensors 24 could be easily related to the flow velocity of the CSF.

The output of the sensors 24 in the shunt evaluation system 20 could be read and processed in any conventional manner. For example, if the internal diameter of the shunt tubing 18 was known, the rate of flow of the CSF could be calculated from the following equation:

$$F = \frac{h π {r}^{2}}{{t}_{1} - {t}_{2}}$$

where F is the flow of CFS through the shunt tubing 18, h is the distance between the two sensors 24, r is the internal radius of the shunt tubing 18 and t₁-t₂ is the time difference between the deflection responses of the two sensors 24.

The following describe different apparatus and methodologies that have been used to monitor, determine or treat body fluid flow, including CSF flow through a shunt.

“A Thermosensitive Device for the Evaluation of the Patency of Ventriculo-atrial Shunts in Hydrocephalus”, by Go et al. (Acta Neurochirurgica, Vol. 19, pages 209-216, Fasc. 4) discloses the detection of the existence of flow in a shunt by placement of a thermistor and detecting means proximate the location of the shunt and the placement of cooling means downstream of the thermistor. The downstream thermistor detects the cooled portion of the CSF fluid as it passes from the region of the cooling means to the vicinity of the thermistor, thereby verifying CSF flow. However, among other things, the apparatus and method disclosed therein fails to teach or suggest an apparatus/method for quantifying the flow of the fluid through the shunt.

In “A Noninvasive Approach to Quantitative Measurement of Flow through CSF Shunts” by Stein et al., Journal of Neurosurgery, 1981, April; 54(4):556-558, a method for quantifying the CSF flow rate is disclosed. In particular, a pair of series-arranged thermistors is positioned on the skin over the CSF shunt, whereby the thermistors independently detect the passage of a cooled portion of the CSF fluid. The time required for this cooled portion to travel between the thermistors is used, along with the shunt diameter, to calculate the CSF flow rate. See also “Noninvasive Test of Cerebrospinal Shunt Function,” by Stein et al., Surgical Forum 30:442-442, 1979; and “Testing Cerebropspinal Fluid Shunt Function: A Noninvasive Technique,” by S. Stein, Neurosurgery, 1980 Jun. 6(6): 649-651. However, the apparatus/method disclosed therein suffers from, among other things, variations in thermistor signal due to environmental changes.

U.S. Pat. No. 4,548,516 (Helenowski) discloses an apparatus for indicating fluid flow through implanted shunts by means of temperature sensing. In particular, the apparatus taught by Helenowski comprises a plurality of thermistors mounted on a flexible substrate coupled to a rigid base. The assembly is placed on the skin over the implanted shunt and a portion of the fluid in the shunt is cooled upstream of the assembly. The thermistors detect the cooled portion of the fluid as it passes the thermistor assembly and the output of the thermistor is applied to an analog-to-digital converter for processing by a computer to determine the flow rate of the shunt fluid.

U.S. Pat. No. 6,413,233 (Sites et al.) discloses several embodiments that utilize a plurality of temperature sensors on a patient wherein a body fluid (blood, saline, etc.) flow is removed from the patient and treated, e.g., heated or cooled, and then returned to the patient. See also U.S. Pat. No. 5,494,822 (Sadri). U.S. Pat. No. 6,527,798 (Ginsburg et al.) discloses an apparatus/method for controlling body fluid temperature and utilizing temperature sensors located inside the patient's body.

U.S. Pat. No. 5,692,514 (Bowman) discloses a method and apparatus for measuring continuous blood flow by inserting a catheter into the heart carrying a pair of temperature sensors and a thermal energy source. See also U.S. Pat. No. 4,576,182 (Normann).

U.S. Pat. No. 4,684,367 (Schaffer et al.) discloses an ambulatory intravenous delivery system that includes a control portion of an intravenous fluid that detects a heat pulse using a thermistor to determine flow rate.

U.S. Pat. No. 4,255,968 (Harpster) discloses a fluid flow indicator which includes a plurality of sensors placed directly upon a thermally-conductive tube through which the flow passes. In Harpster a heater is located adjacent to a first temperature sensor so that the sensor is directly within the sphere of thermal influence of the heater.

U.S. Pat. No. 3,933,045 (Fox et al.) discloses an apparatus for detecting body core temperature utilizing a pair of temperature sensors, one located at the skin surface and another located above the first sensor wherein the output of the two temperature sensors are applied to a differential amplifier heater control circuit. The control circuit activates a heat source in order to drive the temperature gradient between these two sensors to zero and thereby detect the body core temperature.

U.S. Pat. No. 3,623,473 (Andersen) discloses a method for determining the adequacy of blood circulation by measuring the difference in temperature between at least two distinct points and comparing the sum of the detected temperatures to a reference value.

U.S. Pat. No. 3,762,221 (Coulthard) discloses an apparatus and method for measuring the flow rate of a fluid utilizing ultrasonic transmitters and receivers.

U.S. Pat. No. 4,354,504 (Bro) discloses a blood-flow probe that utilizes a pair of thermocouples that respectively detect the temperature of a hot plate and a cold plate (whose temperatures are controlled by a heat pump. The temperature readings are applied to a differential amplifier. Energization of the heat pump is controlled by a comparator that compares a reference signal to the differential amplifier output that ensures that the hot plate does not exceed a safety level during use.

U.S. Patent Publication No. 2005/0171452 (Neff), which is owned by the same assignee as the present application, namely, Neuro Diagnostic Devices, Inc., and which is incorporated by reference herein, discloses a cerebral spinal fluid (CSF) shunt evaluation system that utilizes pairs of temperature sensors, each pair having an upstream and a downstream temperature sensor and whose outputs are analyzed for providing CSF flow rates when an upstream temperature source is applied to the patient.

U.S. Patent Publication No. 2005/0204811 (Neff), which is owned by the same assignee as the present application, namely, Neuro Diagnostic Devices, Inc., discloses a CSF shunt flow measuring system contains upstream and downstream temperature sensors embedded within the wall of a shunt with a temperature source located between the sensors and whose outputs are analyzed for providing CSF flow.

However, there remains a need to quickly and non-invasively, as well as more accurately, determine the flow status or flow rate of a fluid in a subcutaneous tube.

All references cited herein are incorporated herein by reference in their entireties.

### BRIEF SUMMARY OF THE INVENTION

An apparatus for evaluating cerebrospinal fluid (CSF) flow rate or flow status in a CSF shunt applied to the body of a patient for transmitting the CSF between first and second locations of the body. The apparatus comprises: a pad that is placed against the skin of a patient over the location of the CSF shunt, wherein the pad comprises a pair of temperature sensors that are aligned in a first direction to form an upstream temperature sensor (e.g., a fast response thermistor) and a downstream temperature sensor e.g., a fast response thermistor) with respect to the shunt. The pad further comprises a third temperature sensor e.g., a fast response thermistor) that is not aligned in the first direction and each of the temperature sensors generates respective temperature data. The apparatus further comprises a sensor processing device (e.g., a CSF analyzer) that is electrically coupled to the pad for receiving temperature data from each of the temperature sensors, and wherein the sensor processing device uses the temperature data to determine a flow rate or flow status of the CSF through said shunt when a temperature source (e.g., an ice pack or cube) is applied to the pad.

An apparatus for evaluating cerebrospinal fluid (CSF) flow rate or flow status in a CSF shunt applied to the body of a patient for transmitting the CSF between first and second locations of the body. The apparatus comprises: a pad that is placed against the skin of a patient over the location of the CSF shunt, wherein the pad comprises a pair of temperature sensors (e.g., fast response thermistors) that are aligned in a first direction, one of the temperature sensors being positioned over the CSF shunt while the other temperature sensor is not positioned over the CSF shunt, and wherein each of the temperature sensors generates respective temperature data; and a sensor processing device that is electrically coupled to the pad for receiving temperature data from each of the temperature sensors, and wherein the sensor processing device uses the temperature data to determine a flow rate or flow status of the CSF through the shunt when a temperature source (e.g., an ice pack or cube) is applied to the pad.

A method for evaluating cerebrospinal fluid (CSF) flow rate or flow status in a CSF shunt. The method comprises: applying a pair of temperature sensors (e.g., fast response thermistors) against the skin aligned with the CSF shunt to form an upstream temperature sensor and a downstream temperature sensor while simultaneously applying a third temperature sensor (e.g., a fast response thermistor) against the skin in the vicinity of the CSF shunt but not over the shunt; applying a temperature source (e.g., an ice pack or cube) over the CSF shunt and upstream of the pair of temperature sensors for a predetermined period; collecting temperature data after the predetermined period of time (e.g., 60 seconds) has elapsed; subtracting temperature data of the third temperature sensor from each of the temperature data from the pair of temperature sensors to form first and second temperature differences respectively; and determining a flow rate or flow status of the CSF through the shunt from the first and second temperature differences.

A method for evaluating cerebrospinal fluid (CSF) flow rate or flow status in a CSF shunt. The method comprises: applying first and second temperature sensors (e.g., fast response thermistors) against the skin wherein the first temperature sensor is positioned over the CSF shunt and the second temperature sensor is applied against the skin in the vicinity of the CSF shunt but not over the shunt; applying a temperature source over the CSF shunt and upstream of the first temperature sensor for a predetermined period (e.g., 60 seconds); collecting temperature data after the predetermined period of time has elapsed; subtracting temperature data of the second temperature sensor from the temperature data of the first temperature sensor to form a temperature difference; and determining a flow rate or flow status of the CSF through the shunt from the temperature difference.

A method for evaluating cerebrospinal fluid (CSF) flow rate or flow status in a CSF shunt applied to the body of a patient for transmitting the CSF between first and second locations of the body, comprising: applying a first temperature sensor (e.g., a fast response thermistor) at a first location external to the body in a vicinity of the CSF shunt and applying a second temperature sensor (e.g., a fast response thermistor) at a second location external to the body and under which the CSF shunt is located, the first location being upstream of the second location; applying a control temperature sensor (e.g., a fast response thermistor) at a third location under which the CSF shunt is not located but which is aligned with the second temperature sensor, wherein the control temperature sensor provides temperature correction signals representative of a temperature of the exterior of the body; applying a temperature source directly to the first temperature sensor; determining a flow rate or flow status of the CSF through the shunt to provide a determined CSF flow rate or flow status; and adjusting the determined CSF flow rate in accordance with the temperature correction signals to provide a CSF flow rate corrected in accordance with the background temperature.

An apparatus for evaluating cerebrospinal fluid (CSF) flow rate or flow status in a CSF shunt applied to the body of a patient for transmitting the CSF between first and second locations of the body, the apparatus comprising: a first temperature sensor, (e.g., a fast response thermistor) positioned at a first location external to the body and in the vicinity of the CSF shunt and providing first temperature outputs; a second temperature sensor (e.g., a fast response thermistor), positioned at a second location external to the body and under which the CSF shunt is located and providing second temperature outputs, wherein the second location is downstream of the first location; a control temperature sensor, positioned at a third location external to the body and aligned with the second temperature sensor for providing temperature correction signals representative of a temperature of the exterior of the body and forming third temperature outputs; a sensor processing unit (e.g., a CSF analyzer), in communication with the first and second temperature sensors and with the control temperature sensor, the sensor processing unit using said first through said third temperature outputs for determining a flow rate or flow status of said CSF through said shunt when a temperature source is applied directly to the first temperature sensor.

A device for detecting or quantifying fluid flow in a subcutaneous tube of a subject, wherein the device comprises: a temperature sensitive material having properties that change with temperature (e.g., the Mylar® liquid crystal sheets sold by Anchor Optics (AX61161, AX72375, etc.), or by Educational Innovations (LC-3035A, LC-5A, etc.) or by LCR Hallcrest, etc.), and wherein the temperature sensitive material is applied to the skin of the subject over the subcutaneous tube; and wherein a temperature change, applied to the skin at an upstream location of the subcutaneous tube, alters a property of the temperature sensitive material when it (the temperature change) arrives at the material, and wherein the temperature sensitive material provides a correlation between the property change and flow status or flow rate.

A method for detecting or quantifying fluid flow in a subcutaneous tube of a subject, wherein the method comprises: applying a temperature sensitive material having properties that change with temperature (e.g., the Mylar® liquid crystal sheets sold by Anchor Optics (AX61161, AX72375, etc.), or by Educational Innovations (LC-3035A, LC-5A, etc.) or by LCR Hallcrest, etc.), to the skin of the subject over the subcutaneous tube; applying a temperature source to the skin of the subject at an upstream location with respect to the temperature sensitive material; and correlating changes in properties of the temperature sensitive material with different flow rates for indicating flow status or flow rate.

### BRIEF DESCRIPTION OF SEVERAL VIEWS OF THE DRAWINGS

The invention will be described in conjunction with the following drawings in which like reference numerals designate like elements and wherein:

FIG. 1 shows a schematic representation of a prior art cerebral spinal fluid shunt evaluation system for monitoring the fluid flow through the shunt;

FIG. 2 shows a schematic representation of another prior art cerebral spinal fluid shunt evaluation system for monitoring the fluid flow through the shunt;

FIG. 3 shows a schematic representation of a cerebral spinal fluid shunt evaluation system for monitoring the fluid flow through the shunt disclosed in the commonly-owned and co-pending application Ser. No. 10/770,74;

FIG. 4 shows a schematic representation of a circuit suitable for use in the cerebral spinal fluid shunt evaluation system of FIG. 3;

FIG. 5 shows a cerebral spinal fluid flow rate calculation system including the circuit of FIG. 4; and

FIG. 6 shows a graphical representation of the response time of two sensors within the cerebral spinal fluid shunt evaluation system of FIG. 3;

FIG. 7 is a functional diagram of the measurement pad and the CSF analyzer of the present invention;

FIG. 8A is a plan view of the measurement pad and its associated cable and connector;

FIG. 8B is a side view of the measurement pad and its associated cable and connector;

FIG. 8C is an exploded isometric view of an exemplary connector of the measurement pad;

FIG. 9 is an exploded view of the measurement pad of the present invention;

FIG. 10A is a plan view of the top of the measurement pad with exemplary dimensions;

FIG. 10B is a side view of the measurement pad with exemplary dimensions;

FIG. 10C is a plan view of the bottom of the measurement pad with exemplary dimensions;

FIG. 11 is an isometric view of a hand-held CSF analyzer that electrically couples to the measurement pad;

FIG. 12 shows how the measurement pad is placed on the patient's skin while being located over the shunt tube (shown in phantom) beneath the skin and electrically coupled the to the CSF analyzer (not shown);

FIG. 13 is a specification sheet of an exemplary fast response thermistor for use in the measurement pad of the present invention;

FIG. 14A depicts exemplary temperature profiles of test data where the control sensor data is subtracted from the proximal sensor data and from the distal sensor data;

FIG. 14B depicts exemplary raw temperature data from each of the three temperature sensors;

FIG. 15 is a functional diagram of an alternative measurement pad using a plurality of proximal temperature sensors and a control sensor but no distal temperature sensor;

FIG. 16A is a top isometric view of an alternative embodiment of the measurement pad;

FIG. 16B is a bottom isometric view of the measurement pad of FIG. 16A;

FIG. 16C is an exploded view of the alternative measurement pad;

FIG. 16D is a plan view of the top of the measurement pad with exemplary dimensions;

FIG. 16E is a side view of the measurement pad with exemplary dimensions;

FIG. 16F is a plan view of the bottom of the measurement pad with exemplary dimensions;

FIG. 16G is an isometric view of the lower portion of the alternative measurement pad; and

FIG. 16H is an isometric view of the reverse side of the lower portion of the alternative measurement pad;

FIG. 17 shows a schematic representation of the cerebral spinal fluid shunt evaluation system of the present invention for monitoring the fluid flow through the shunt whereby a temperature source is positioned directly over one of the temperature sensors;

FIG. 18 is a plan view depicting the relative positions of the various temperature sensors in the cerebral spinal fluid shunt evaluation system of the present invention;

FIG. 19 is a plan view depicting another cerebral spinal fluid shunt evaluation system of the present invention which uses a temperature sensitive material (e.g., a film) that is applied to the skin of a subject having a subcutaneous tube (shown in phantom);

FIG. 20 is a partial cross-sectional view of the invention of FIG. 19 showing the relative positions of the present invention with regard to the subcutaneous tube; and

FIG. 21 is an exemplary grid used for flow rate estimation using the invention of FIGS. 19-20.

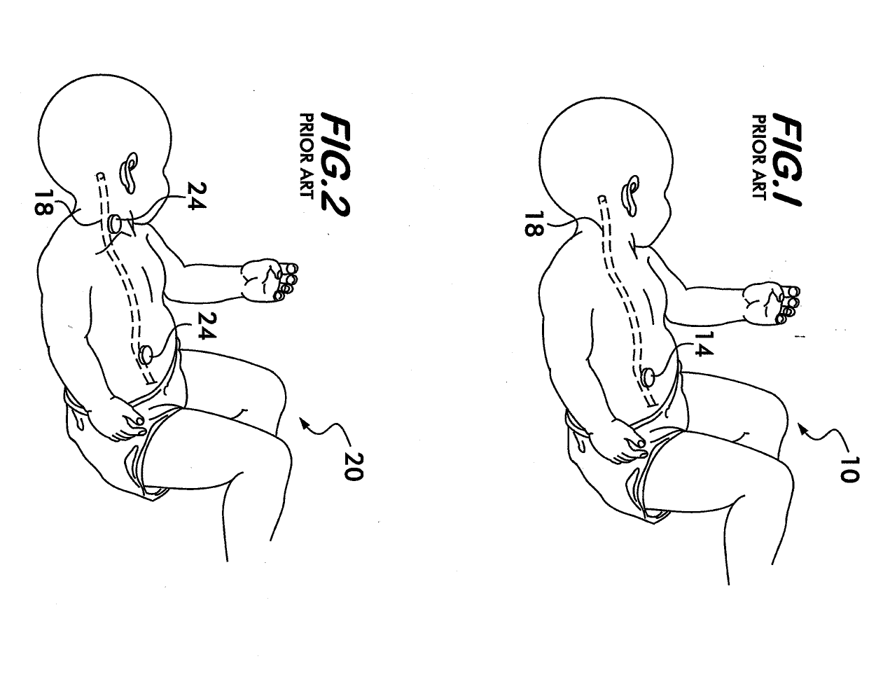

### DETAILED DESCRIPTION OF THE INVENTION

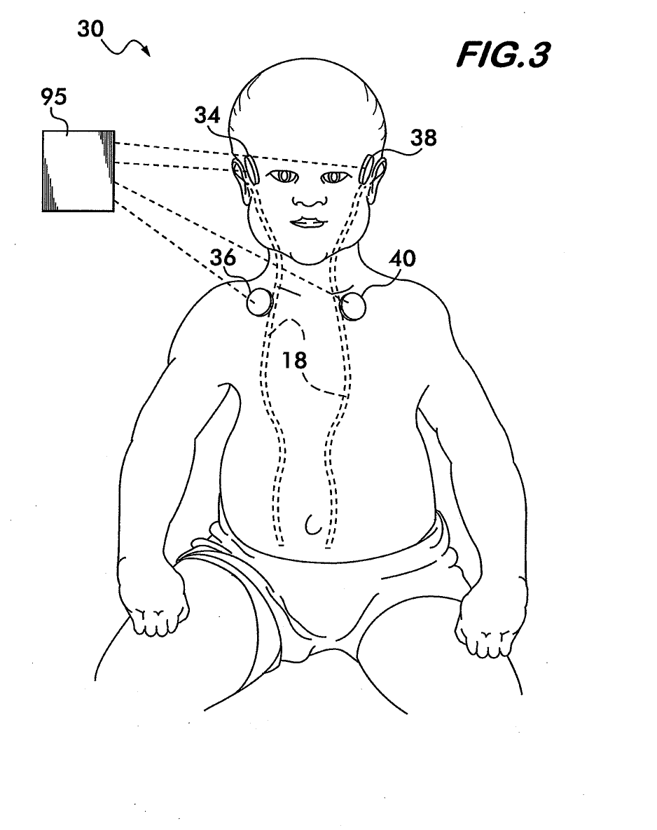

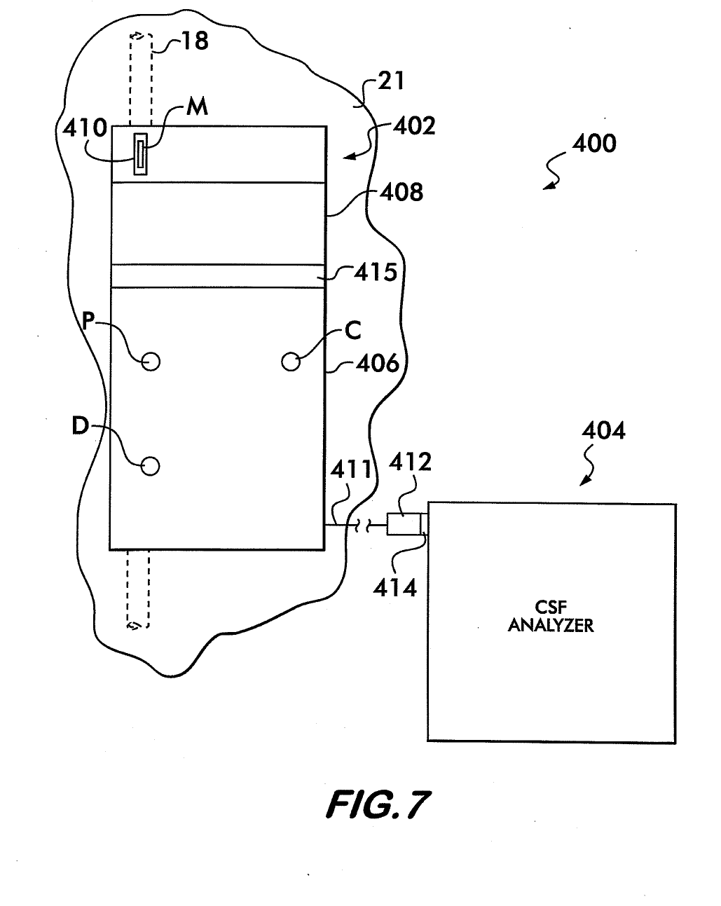

The invention of the present application involve improvements over an invention of an earlier application, namely, application Ser. No. 10/770,754 (U.S. Patent Publication No. 2005/0204811 (Neff)), and as such the present application is a continuation-in-part of application Ser. No. 10/770,754. Therefore, before discussing the invention of the present application (FIGS. 7-21), Applicants will first discuss the invention of application Ser. No. 10/770,754 (FIGS. 3-6).

Referring now to FIG. 3, there is shown a CSF shunt evaluation system 30. The CSF shunt evaluation system 30 is provided with four sensors 34-40 disposed at predetermined locations on the body of the patient for determining the existence of CSF flow through the shunt tubing 18, and determining the flow and the flow rate of the CSF through the shunt tubing 18. Additionally, the placement of the four sensors 34-40 in the CSF shunt evaluation system 30 is adapted to permit the calculation of error signals due to background effects such as body temperature and ambient temperature. The error signals within CSF shunt evaluation system 30 can be used to provide a more accurate determination of the CSF flow rate through the shunt tubing 18.

In the method of the invention a sensor 34 is placed over the shunt tubing 18 in the vicinity of an ear of the patient for providing an electrical output signal representative ofthe temperature of the CSF near the vicinity of the cooling of the CSF of the patient. A sensor 36 is placed over the shunt tubing 18 in the vicinity of the clavicle of the patient for providing an electrical output signal representative of temperature of the CSF therebelow.

Preferably the sensors 34, 36 can be disposed as close as possible to each other, as long as they are placed in an area where the shunt tubing 18 is substantially close to the surface of the body. The shunt tubing 18 is usually sufficiently close to the surface behind the pinna and on the neck. It is also close to the surface over the clavicle, which is often approximately fifteen centimeters from the pinna. Thus, in one preferred embodiment of the invention the spacing between the sensors 34, 36 can be approximately fifteen centimeters or less. Furthermore, in one preferred embodiment the sensors 34, 36 can be placed as close together as approximately three centimeters.

The sensors 38, 40 are placed on the opposite side of the body of the patient in locations substantially symmetrically with the sensors 34, 36. Thus, the sensor 38 is placed in the vicinity of the ear opposite the ear where the sensor 34 is disposed. The sensor 38 is placed in the vicinity of the clavicle opposite the clavicle where the sensor 36 is disposed. The sensors 38, 40 thus provide electrical output signals representative of background conditions such as the body temperature of the patient and the ambient temperature. The output signals from the sensors 38, 40 permit control readings to be performed by the CSF evaluation system 30 for error correction of the flow rate calculations that can be obtained using the sensors 34, 36.

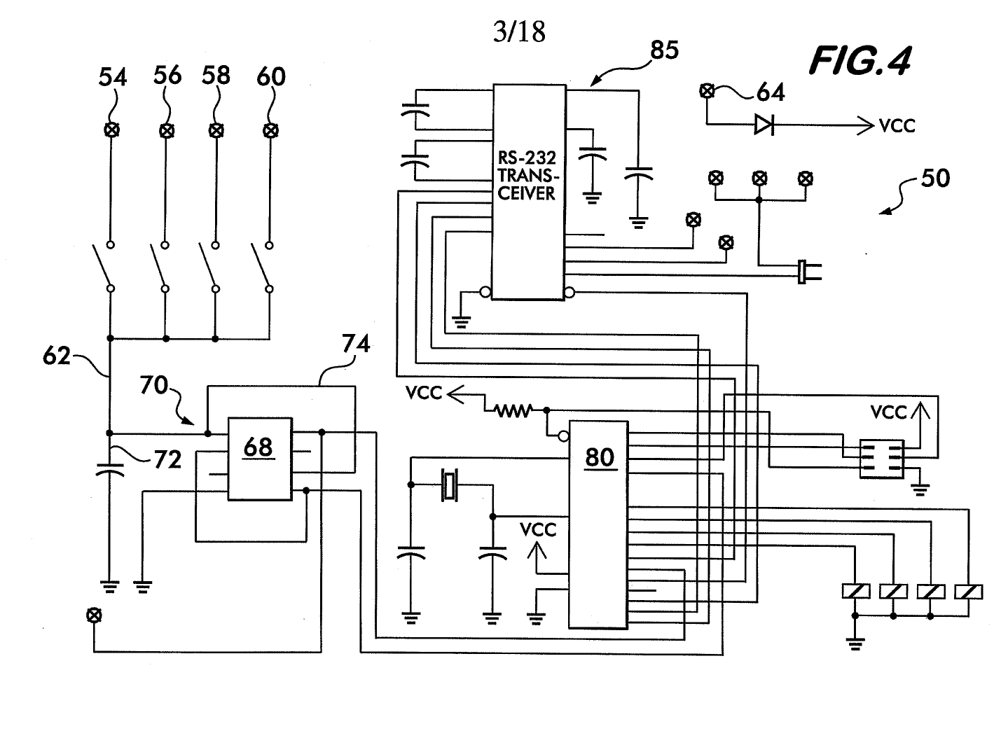

Referring now to FIG. 4, there is shown a schematic diagram of the shunt evaluation system circuitry 50. The shunt evaluation system circuitry 50 can be used for receiving and processing the electrical output signals provided by the sensors 34-40 of the CSF shunt evaluation system 30. The shunt evaluation system circuitry 50 processes the signals from the sensors 34-40 to provide further electrical signals representative of the temperatures of the sensors 34-40 to permit the determination of the flow rate of the CSF through the shunt tubing 18 as previously described.

The output signals of the sensors 34-40 applied to the body of the patient are received at the input lines 54-60 of the evaluation system circuitry 50. In one preferred embodiment of the invention, the signals received on the input lines 54-60 can be sequentially switched onto a common input line 62 of a general purpose precision timer 68. Additionally, in an alternate embodiment of the invention, the signals on the input lines 54-60 can be applied to an analog-to-digital converter (not shown) to provide digital signals representative of the output of the sensors 34-40 suitable for processing within the evaluation system circuitry 50.

The precision timer 68 of the evaluation system circuitry 50 that sequentially receives the signals from the sensors 34-40 is adapted to operate as a relaxation oscillator circuit 70 having a varying output frequency related to a varying RC time constant. The precision timer 68 within the relaxation oscillator circuit 70 can be the well known ICM7555 or any other equivalent device.

The precision timer 68 is coupled to a capacitor 72 and to the common input line 62 of the four input lines 54-60. Each of the sensors 34-40 coupled in sequence to the common input line 62 operates as a variable resistor whose resistance varies with a sensed temperature as previously described. The sequential coupling of the sensors 34-40 to the capacitor 72 permits RC time constant within the relaxation oscillator circuit 70 to vary when the sensors 34-40 sense different temperatures. Thus, the varying RC time constant results in varying frequencies of oscillation for the relaxation oscillator circuit 70 that correspond to the varying temperatures sensed by the sensors 34-40.

When the relaxation circuit 70 of the shunt evaluation system circuitry 50 oscillates, a battery 64 charges the capacitor 72 according to the resistance of the sensor 34-40 coupled to the capacitor 72. This causes the voltage across the capacitor 72 to rise. When the voltage across the capacitor 72 rises to a predetermined level, the precision timer 62 triggers. The triggering of the precision timer 68 causes the capacitor 72 to discharge through the precision timer 62 by way of the line 74, thereby completing one cycle of the relaxation oscillator 70. The time period it takes for the capacitor 72 to charge to the predetermined voltage level and trigger is determined by the amount of charging current, and thus the amount of resistance, of the sensor 34-40 coupled to the common input line 62. Thus, the oscillation frequency of the relaxation oscillator 70 is determined by the resistance, and thus the temperature, of the active sensor 34-40.

The use of the relaxation oscillator 70 for obtaining an electrical signal representative of the resistance of the sensors 34-40 suitable for algorithmic processing is believed to be easier and less expensive than the use of an analog-to-digital converter for this purpose. Additionally, use of the relaxation oscillator 70 is believed to be more noise resistant than an analog-to-digital converter. Furthermore, the relaxation oscillator 70 uses less power than an analog-to-digital converter uses.

The frequency signal output of the precision timer 68 is applied to an input pin of a microprocessor 80 of the shunt evaluation system circuitry 50. The microprocessor 80 can be an AT90S2313 8-bit microcomputer, or any other microprocessor known to those skilled in the art. In addition to controlling the sequential switching of the sensors 34-40 onto the common input line 62, the microprocessor 80 can operate as a frequency counter to determine a frequency value in accordance with the oscillation frequency of the relaxation oscillator 70. The frequency value determined by the microprocessor 80 is provided as an output of the shunt evaluation system circuitry 50 on an output bus 85. The output bus 85 can be coupled to a conventional RS-232 transceiver. In keeping with the system of the present invention, the output frequency value can also be provided on a parallel bus.

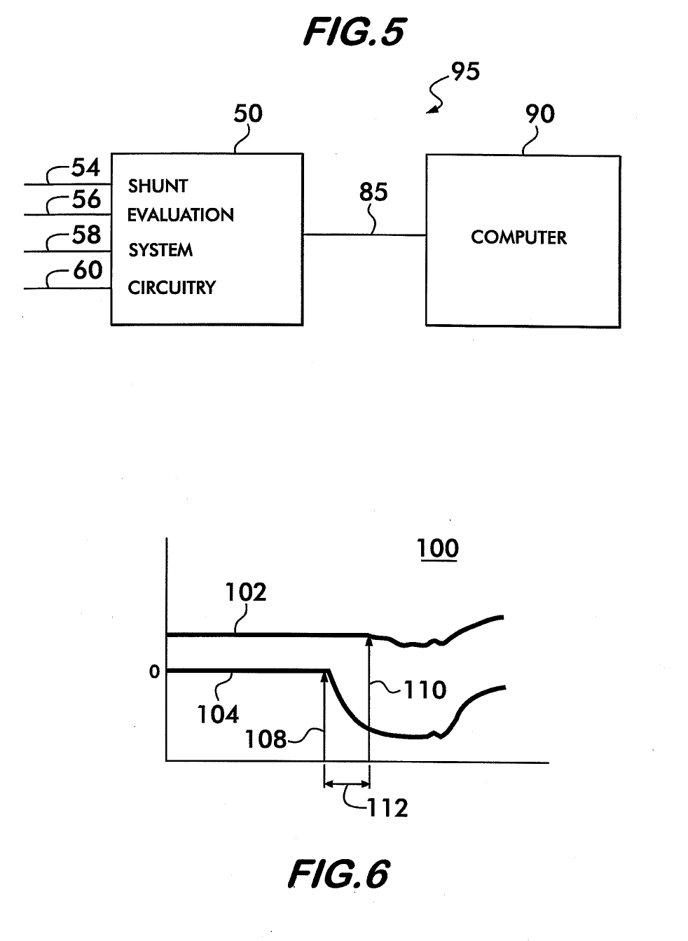

Referring now to FIG. 5, there is shown the CSF flow rate calculation system 95. Within the CSF flow rate calculation system 95, a computer 90 receives the frequency values determined by the shunt evaluation system circuitry 50 by way of the output bus 85. When the frequency values are received, the computer 90 performs calculations on them in order to determine the flow rate of the CSF through the shunt tubing 18 of the system 30 under the control of a stored program. Signals from the sensors 34, 36 can be used by the computer 90 to calculate the flow rate through the shunt tubing 18 as previously described. For example, the flow rate calculation set forth above with respect to the CSF shunt evaluation system 20 can then be used to determine the CSF flow rate in accordance with the determined time difference 112. Signals from one or both of the sensors 38, 40 can be used to determine an error correction signal representative of background conditions for use in correcting the calculations performed on the signals from the sensors 34, 36.

Referring now to FIG. 6, there is shown a graphical representation 100 of the response times of the sensors 34, 36 within the CSF flow rate calculation system 95. The inflection point of the temperature inflection curve 104, representing the temperature of the sensor 34, occurs first since the cooled CSF reaches the sensor 34 first. The curve 104 inflection point occurs at time 108. At a time thereafter, varying according to the flow rate of the CSF, the inflection point of the curve 102 occurs. Curve 102 represents the temperature of the sensor 36. The temperature infection curve 102 inflection point occurs at time 110. A skilled practitioner, preferably a neurosurgeon, determines the time difference 112 between the inflection points 108, 110.

In the error correction protocol, the skin temperature at the location 38, which is the mirror-image of the location 34, is subtracted from the skin temperature at the location of sensor 34. Additionally, the skin temperature at the location of sensor 40 is subtracted from the skin temperature at the location of sensor 36. These subtractions correct for global skin temperature changes such as changes due to environment and physiology, for example excitement, attention and pain, and provide error correction for adjusting the flow rate to provide a corrected CSF flow rate.

Using the correct (subtracted) temperature curve makes it possible in a realistic clinical situation to accurately detect inflection points as the cooled CSF passes under the thermistors. When the time of the inflection pints 108, 110 and the time difference between the inflection points 108, 110 are determined the flow calculation can be performed in substantially the same manner as the flow calculations of the prior art.

For example, in one embodiment of the invention the software providing graphical representation 100 displays on the screen two temperature inflection curves 102, 104 one for the proximal (shunt temperature minus control temperature) pair of thermistors and one for the distal (shunt temperature minus control temperature) pair. The operator can use a mouse to move two vertical bars to the inflection points 108, 110.

The software can provide a window showing the times corresponding to the inflection points 108, 110 selected and prompting the operator for the diameter of the tubing. Since only two diameters are in common clinical use, the window can allow a choice between these two in the preferred embodiment. The software then calculates the flow rate from the time difference and the diameter.

In view of the foregoing, the embodiments of the present invention are now discussed.

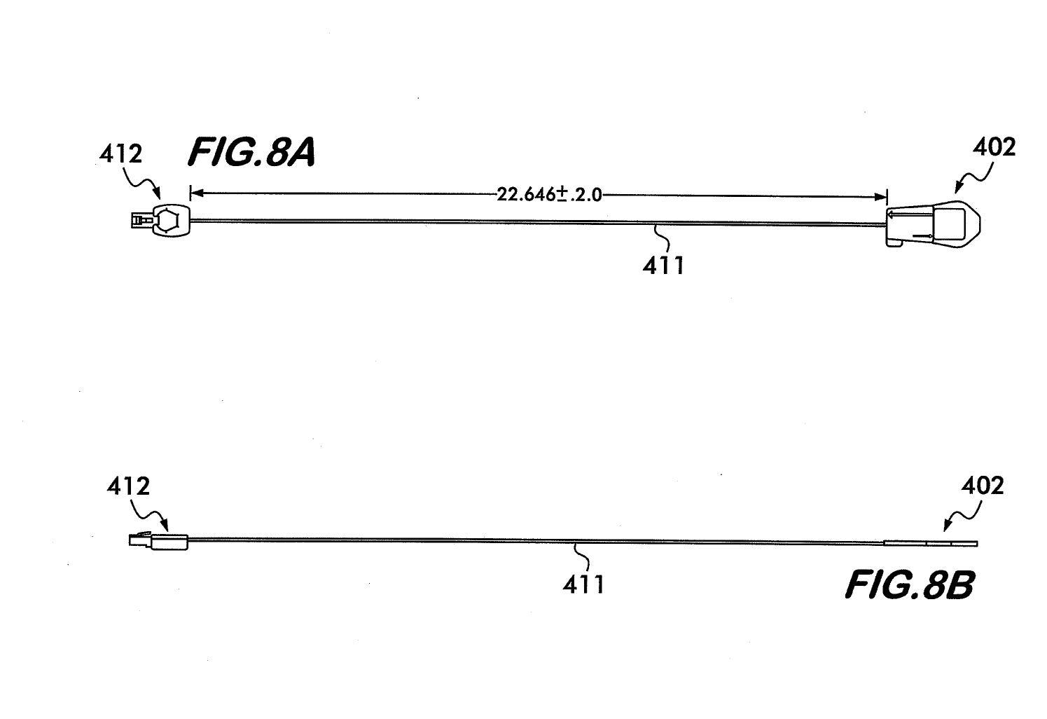

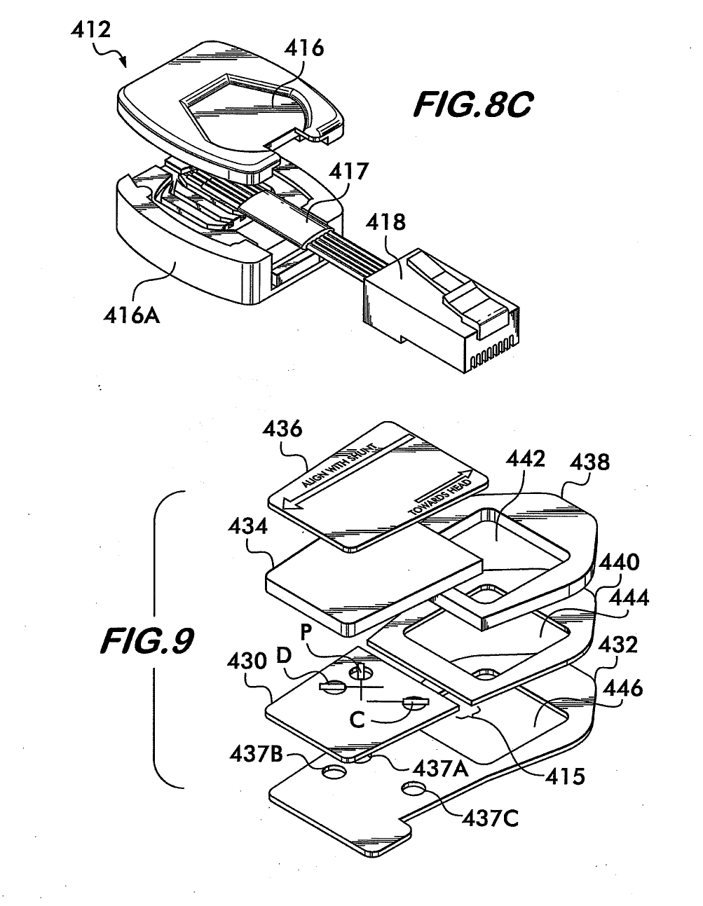

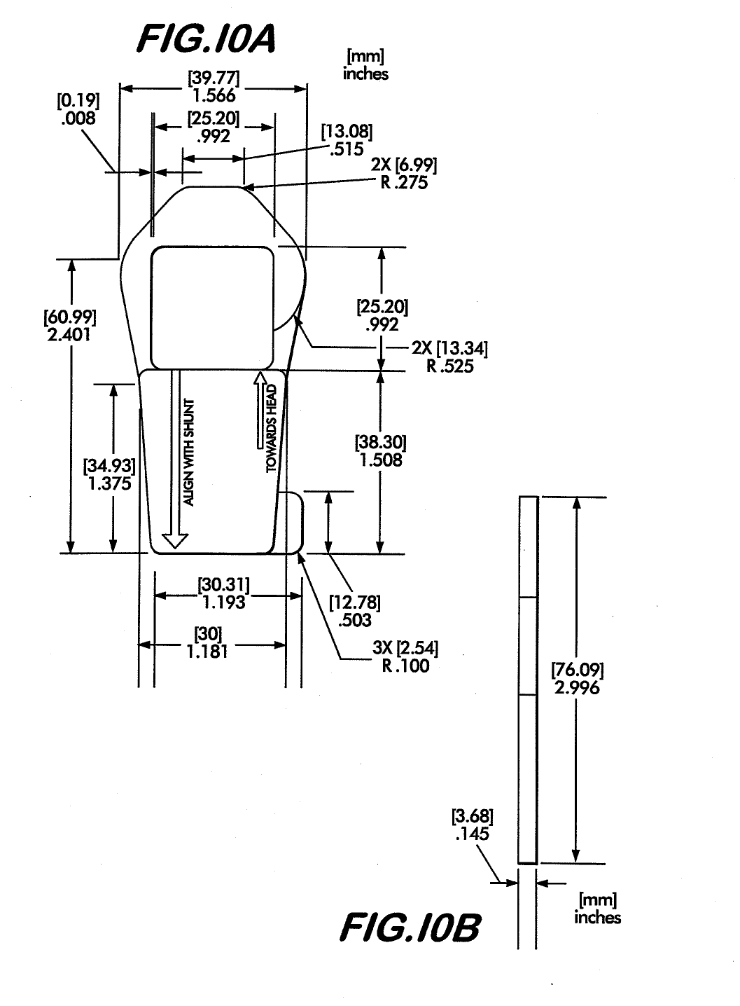

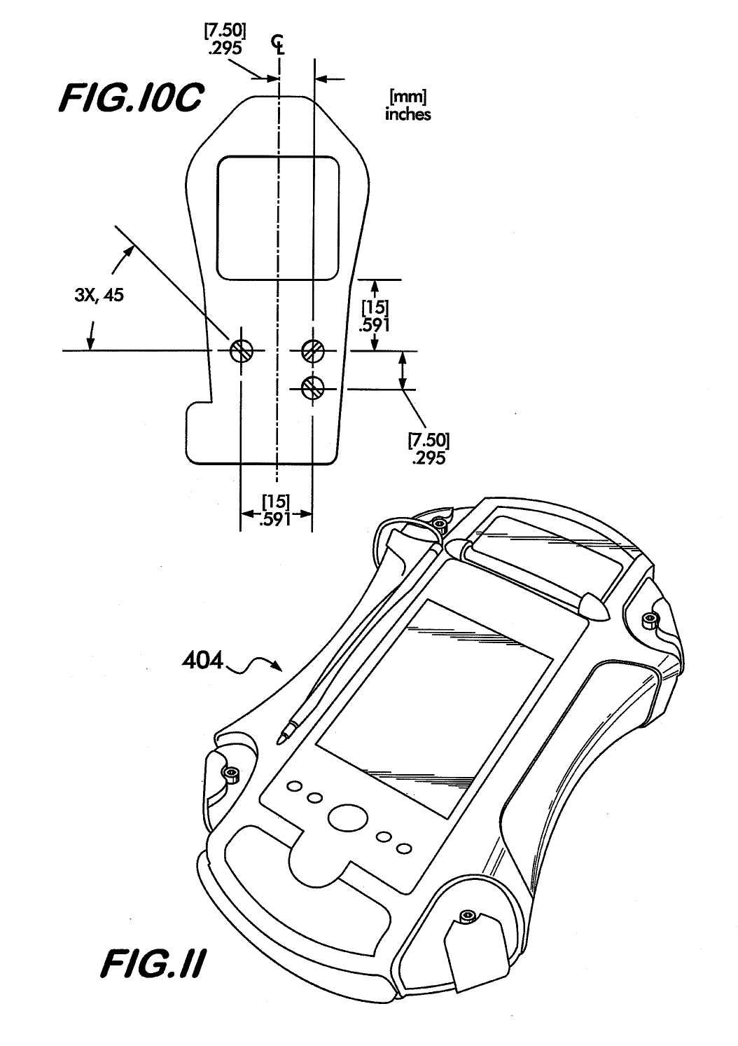

Referring now to FIG. 7, there is shown a functional diagram of the system and method of the present invention 400. In particular, the invention 400 comprises a thermal flow measurement pad 402 (see also FIGS. 9 and 10A-10C) which is in electrical communication with an analyzer 404 (see FIGS. 7 and 11), also known as a sensor processing device (e.g., a processor with I/O) and in many ways is similar to the CSF flow rate calculation system 95 of application Ser. No. 10/770,754. As will be discussed in detail later, the measurement pad 402 comprises a plurality of sensors, such as thermistors, which are maintained in the correct relative geometries by the measurement pad 402. The analyzer 404 also provides the sensor excitation. The measurement pad 402 improves the performance of methods for thermal measurement of CSF flow in implanted shunts. In particular, the measurement pad 402 provides substantially greater accuracy and repeatability. Additionally, the measurement pad 402 makes such flow and flow rate measurements substantially easier and more convenient. As can be seen in FIG. 7, one of the key distinctions ofthe present invention 400 with respect to the previously-described CSF shunt monitoring systems, is that the plurality of sensors are localized within the measurement pad 402. Furthermore, the number of sensors is reduced in the present invention 400, as will be discussed shortly. As shown in FIGS. 7, 8A and 8B, the measurement pad 402 includes an electrical cable 411 having a connector 412 that couples to a mating connector 414 in the CSF analyzer 404. By way of example only, the connector 412 comprises a housing bottom 416A and a top plate 416 that capture a flat modular cable 417 which terminates in an RJ-45 connector 418 (see FIG. 8C).

It should be understood that the dimensions provided in FIGS. 10A-10C are by way of example only and are not meant to limit the invention to those dimensions.

In one preferred embodiment (FIG. 7) of the invention, the measurement pad 402 is provided with a first pad portion 408 (e.g., at least one clear window) in order to permit accurate placement of the measurement pad 402 and the uniform application of a temperature source, e.g., a cooling means such as an ice cube or pack. It is preferable to use a “plastic ice” cube (which contains water) which avoids or minimizes leaking when compared to an ice cube. To use this embodiment of the measurement pad 402, the shunt tube 18 (which is positioned below the patient's skin) can be located by the physician and the patient's skin can be marked M with a pen or other marking device in order to indicate the location of the shunt tube 18. The measurement pad 402 is then manipulated until the mark M appears in an aperture 410, as shown in FIG. 7 (or, alternatively, a mark on the skin can be aligned with other indicia on the measurement pad 402; see the indicia on the label 436 in FIG. 9). This correct positioning permits an upstream or proximal thermistor P and a downstream or distal thermistor D in the measurement pad 402, viz., in a second pad portion 406, to be positioned over the shunt tube 18. A third thermistor, which acts a control thermistor C is also provided in the measurement pad 402. This thermistor C is positioned in the pad 402 so that when the pad 402 is placed against the skin, it is located in the vicinity of the shunt tubing 18 but not located over the shunt tubing 18, as are the other thermistors P and D. It is preferable to have the control thermistor C aligned with the proximal thermistor P in a direction that is generally perpendicular to the shunt tubing 18. Among other things, the control thermistor C is useful if the cold wave from the cooling of nearby skin by the cooling means reaches the test thermistors P and D and interferes with their measurements. These thermistors are located in a lower portion 406 of the measurement pad 402. The clear window 408 on the measurement pad also allows for accurate placement of the pad 402 over the pen mark or other mark M and therefore over the shunt tube. Alternatively, notches, holes, clear material or any other types of markers or devices for assisting in the placement of the measurement pad 402 over the mark M can be used. The analyzer 404 uses the output of these thermistors to provide an accurate and repeatable determination as to flow/no flow and flow rate.

As will be discussed in detail later, it has been found that the accurate and repeatable determination of flow/no flow and flow rate can be obtained without the need for the distal thermistor D, i.e., use ofthe proximal thermistor P and the control thermistor C are all that is actually needed.

The first and second pad portions 408 and 406 are preferably not contiguous and are preferably separated by a gap or by insulation 415, as shown in FIG. 7.

As shown most clearly in FIG. 10C, an optimal distance (e.g., 15 mm) exists between the ice or other cooling means and the proximal thermistor P of the measurement pad 402. Furthermore, the accuracy of the test results provided by the measurement pad 402 is enhanced by tight and precise distances between the cooling means and the thermistors P, C and D. Therefore, the location of the window 408 on the measurement pad 402, relative to the proximal thermistor P, is adapted to reliably provide the optimal distance between the cooling means and the proximal thermistor P when the cooling means is placed on the window 408 and centered. Thus, when the cooling means is placed on the window 408 of the pad, it is located at the optimal distance from the thermistors P, C and D. The uniform or symmetric application of the cooling pulse is important for the detection mechanism to work properly and thus a variety of window 408 shapes are encompassed by the present invention 400. One exemplary configuration is to have a circular-shaped window 408 (e.g., a 1 inch radius). When the cooling means is applied, it is applied for 60 seconds and then removed from the window 408. However, before applying the cooling means, a measurement pad 402 “warm up” period (e.g., a few minutes) occurs, i.e., the pad 402 is applied to the skin and permitted to reach the skin temperature. Once that skin temperature is achieved, then the cooling means is applied for 60 seconds. It has also been determined that the amount of pressure applied to the cooling means when placed in the window needs to be uniform.

The measurement pad 402 can be insulated in the region around the top of the pad 402 and the window 408 so that the cooling means can slightly overlap the edge without shortening the effective ice-to-thermistor distance. Thus, if a cold pack or an ice pack or some other cooling means without a clean edge is used, the cooling means could be placed at the edge of the measurement pad, or slightly overlapping the edge. The window 408 serves the purpose of insulating the thermistors from the cooling means in addition to its role in insuring the optimal placement of the cooling means and preventing melting ice from dripping onto the patient. It is important to have proper thermal separation of the ice window 408 to prevent thermal conduction to the thermistors other than via the CSF flow. Furthermore, the window can prevent melting ice from dripping onto the patient. In particular, as shown in FIG. 9, the measurement pad 402 comprises a polyimide layer 430 which contains the thermistors P, C and D. This layer 430 is positioned upon an adhesive bottom layer 432. Positioned over the polyimide layer 430 is a Poron MSRVS foam 434 and to which a measurement pad label 436 is applied. The label 436 may comprise indicia for helping the user to align the thermistors P and D over the shunt tubing 18. Apertures 437A, 437B and 437C in the adhesive bottom layer 432 permit a sensing path for the respective thermistors P, D and C. An absorbing layer 438 is positioned over an insulator layer 440 which is placed upon the adhesive bottom layer 432; this not only provides drip protection but can enhance patient comfort as well as prevent cold water from leaking underneath the pad. The window 408 is formed by respective apertures 442, 444 and 446 in the absorbing layer 432, insulator layer 440 and the adhesive bottom layer 432. A gap 415 acts to insulate the window 442 and the thermistors P, C and D and provides thermal isolation. In addition, the Poron MSRVS foam avoid accidental cooling of the thermistors directly from the window 408.

In addition, the positioning between the proximal thermistor P and the distal thermistor D is also important and its optimal distance is approximately 15 mm.

Thermal grease can be used to enhance thermal conduction between the thermistors P, C and D and the patient's skin. The thermal grease can be applied during assembly of the measurement pad 402 or it can be applied at the time the measurement pad 402 is used, for example, with a pen-like device. This allows the user to simultaneously mark the shunt position on the skin and provide conductive grease along the shunt.

Software geared to head space distance and specialized adhesive can be provided for the measurement pad 402. A covering can be provided on the measurement pad 402 and, after the covering is removed, the pad can be placed in any position. After a period of time, the adhesive fails. Under these circumstances, the measurement pad 402 cannot be reused. It is preferable to make the measurement pad 402 a one-time use device and include an interlock that prevents the re-use of the measurement pad 402. As mentioned earlier (see FIGS. 7, 8A and 8B), the measurement pad 402 includes an electrical cable 411/connector 412 that couples to a mating connector 414 in the CSF analyzer. The electrical connector 412 may include an integrated circuit that detects the use and should the connector 412 ever be reconnected to a CSF analyzer 404, the CSF analyzer 404 provides an indication to the operator of the prior use and prevents the test from commencing. In particular, each measurement pad 402 may contain an electronic code which matches codes logged into the accompanying CSF analyzer 404 (FIG. 11). Thus, the CSF analyzer 404 can be programmed to operate only with selected measurement pads 402. For example, the thermistors may themselves contain the code or information.

The measurement pad 402 can be provided with a feature that indicates the precise time the cooling means is positioned on the window 408 or the head. For example, a further thermistor or a switch can be provided in the vicinity of the cooling area.

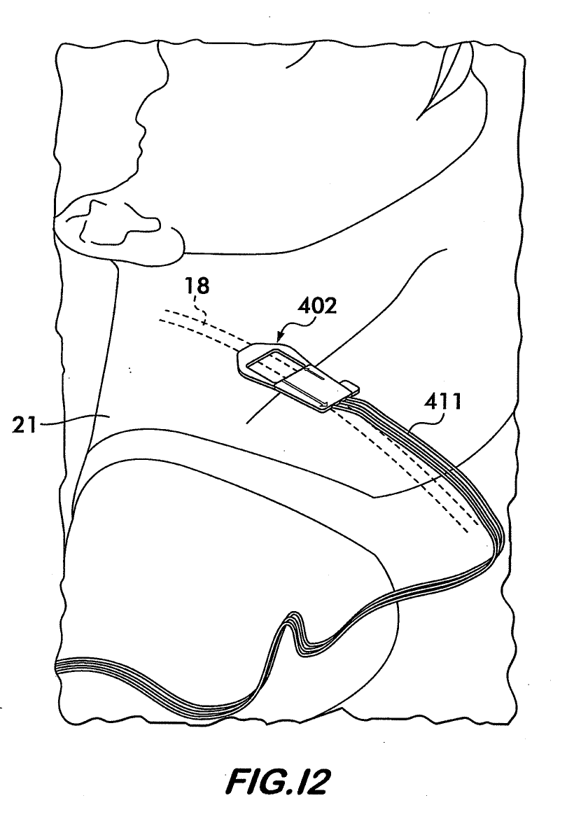

In either measurement pad embodiment 402/402A (see FIG. 15), it should be understood that the type of thermistor used for the proximal P, control C and distal D thermistors must be fast response thermistors, i.e., a time constant of <5 seconds. This is important because the thermistor must be able to track the actual temperature without an appreciable time lag. By way of example only, FIG. 9 is specification sheet of an exemplary fast response thermistor that can be used for the thermistors P, D and C in the measurement pad 402. As can be seen from FIG. 13, the MA100 Catheter Assembly has a thermal response time in still water of 2.0 seconds. Another exemplary thermistor is the GE NTC thermistor.

It has been found that upon initial application of a cooling means to the skin, the temperature in the vicinity may actually rise and then fall, possibly due to the sympathetic system reacting to the cooling means and attempting to maintain equilibrium (hereinafter known as the “flushing effect”). Such a phenomena is not taken into account by the prior CSF shunt mechanisms because the control sensor is located so far away from the cooling means application site. In contrast, in the present invention 400, with the control thermistor C located relatively close to the proximal thermistor P, the control thermistor C also experiences this phenomena of a temperature rise then fall and thereby provides an accurate read of the cooling means pulse.

It has also been found that it is ideal to have the patient placed in a supine position for a predetermined period of time (e.g., 5 minutes). This permits the ventricle to refill. Once the measurement pad 402 is warmed up and the testing is ready to begin, the patient is then permitted to come to a sitting position to permit gravity to accelerate the CSF flow. Attempting to conduct the test on patient who has been in a standing or seated (upright) position drains the cranium and results in a no flow condition, which is normal.

The following discussion is directed to the operation of the present invention 400 which uses all three thermistors, P, D and C. However, as mentioned previously, it should be understood that it is within the broadest scope of the present invention to eliminate the distal thermistor D.

As discussed previously, the present invention 400 is provided with the two thermistors P and D separated by a predetermined distance (e.g., 15 mm) for determining the existence of CSF flow through the shunt tubing 18, and determining the flow status (i.e., flow or no flow) and the flow rate of the CSF flow F through the shunt tubing 18. The upstream or proximal sensor P measures the temperature as the cooling pulse passes from the cooling means and into the CSF in the shunt tubing 18. The downstream or distal thermistor D measures the temperature over the shunt tubing 18 at the predetermined distance from the proximal sensor P. Also, the control thermistor C is used, along with the proximal and distal thermistors P and D, to permit the calculation of error signals due to background effects such as body temperature and ambient temperature. The error signals within CSF shunt evaluation system 400 can be used to provide a more accurate determination of the CSF flow status or rate through the shunt tubing 18. It is this conduction through the skin that is detected by the control thermistor C. The alignment assures that the proximal thermistor P detects the temperature delta via the shunt tubing 18 while the temperature delta propagated via the skin is detected by the control sensor C. The control thermistor C thus provides electrical output signals representative of the detected temperature delta transmitted through the skin. The output signals from the control thermistor C permits control readings to be performed by the CSF evaluation system 400 for error correction of the flow rate calculations that can be obtained using the thermistors P and D. All of the thermistors P, D and C must be equalized for static and dynamic responses.

In accordance with the temperature profiles shown in FIG. 3A, the depth of the temperature profiles is a function of the CSF flow, i.e., the faster the CSF flow, the deeper the “dip” in the temperature profile. The CSF evaluation system 400 operates in a similar manner but with the additional improvements.

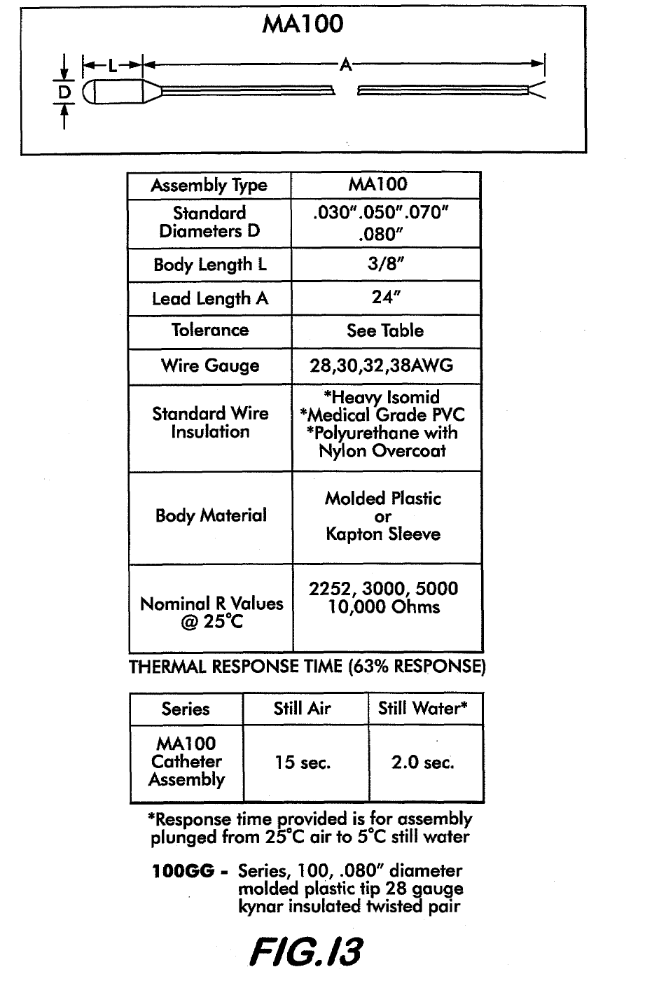

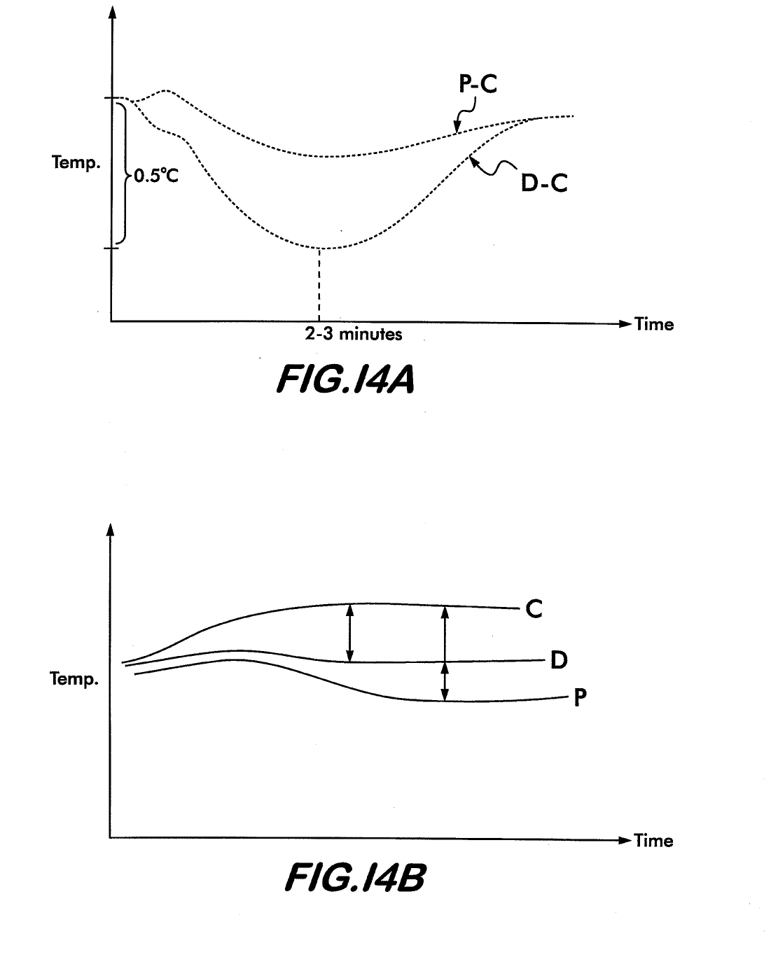

Using a sampling rate of approximately 10 samples/sec (down to a minimum of 1 sample/sec), the three thermistors begin obtaining temperature data once the test begins (see FIG. 14A). As shown in FIG. 14B, the CSF analyzer 404 determines the temperature profile of P-C and D-C. The subtraction of the control thermistor C is critical because it is subjected to the same effects as the proximal and distal thermistors P and D. By doing this, the unwanted effects (e.g., chilling of skin, flushing effect, etc.) are cancelled out of the temperature data. The positioning of the control thermistor C is such that it is “close enough” to detect the cold pulse through the skin/tissue but “far enough” away from the shunt tubing 18 to not detect the cold pulse being propagated through the CSF in the shunt tubing 18. A typical temperature “trough” (see FIG. 14B) is approximately 2-3 minutes for a test run of approximately 9 minutes, with the algorithm itself (the CSF analyzer 402) taking approximately 6 minutes.

In order to provide accurate readings, it is necessary to verify certain criteria, for example:
1) verifying that the temperature data reaches a predetermined value within a certain time limit (e.g., within 5 minutes). If it takes more than 5 minutes to reach that predetermined value, there is something incorrect in the test setup. For example, a typical maximum temperature differential of 0.5° C. (D−C) is achieved in approximately 2-3 minutes (see FIG. 14B).
2) Checking the smoothness of the curves. A spike in the data is most probably an undesirable movement of the measurement pad 402.
3) Proximal thermistor P data amplitude must be greater than distal thermistor D data amplitude;
4) A threshold ratio regarding the proximal thermistor data and the distal thermistor data should be satisfied:

$$thres hold ratio = \frac{P - C}{D - C} = 1.5 to 4.0$$

Where P−C data≧0.2° C. indicates CSF flow and D−C ≧0.1° C. also indicates CSF flow for a given time frame.
5) Slope checks (temperature data must decrease then rise as shown in FIG. 14B).
6) Integral checks-using the area under the curve in relation to flow rate.
7) Gradients (verifying the gradients in the temperature drops in relation to flow rate).

As mentioned previously, it is within the broadest scope of the present invention to eliminate the presence of the distal thermistor D. Thus, in such an embodiment, the CSF analyzer 402 need only analyze the P−C data. In fact, it is desirable to have a plurality of proximal thermistors P1-Pn in the measurement pad 402A, as shown in FIG. 15. The advantage of this is that it widens the test area, reduces mis-aligned thermistors (i.e., not positioning both the proximal thermistor P and the distal thermistor D directly over the shunt tubing 18; for example, the measurement pad 402 could be tilted) and thermistor redundancy. With regard to the latter feature, the CSF analyzer 404 can monitor the temperature data from all of the proximal thermistors P1-Pn and select the one that has the maximum P−C values.

It should be further noted that a plurality of distal thermistors D1-Dn could also be used to also widen the distal test area, where distal thermistor data is desirable.

It should also be noted that it is within the broadest scope of the present invention to include a recharging stand for the CSF analyzer 402 (when it is a hand-held device) that can communicate with a personal computer.

Another embodiment of the measurement pad 402 may include a built-in Peltier device which would eliminate the need for an external cooling means. Alternatively, the cooling means could be separate from the sensor patch but shaped to integrate with the measurement pad 402 for the test. Thus, the Peltier device can be re-used while the measurement pad 402 remains a discardable device.

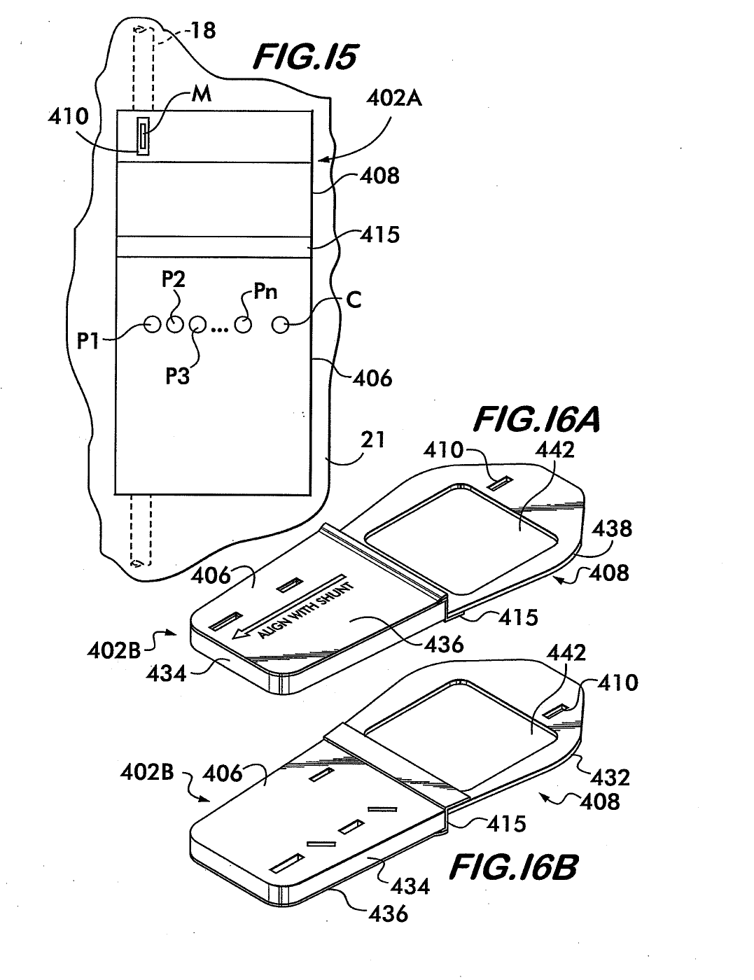

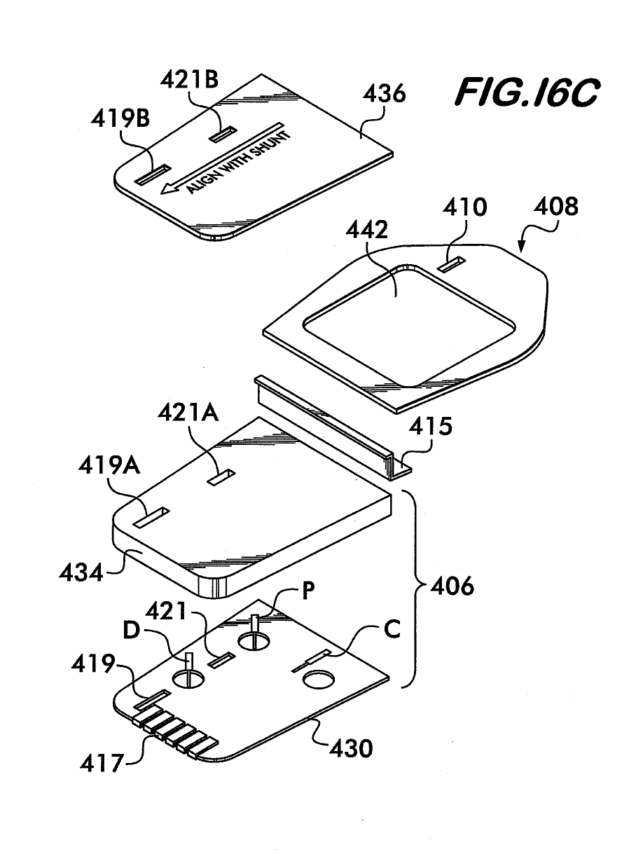

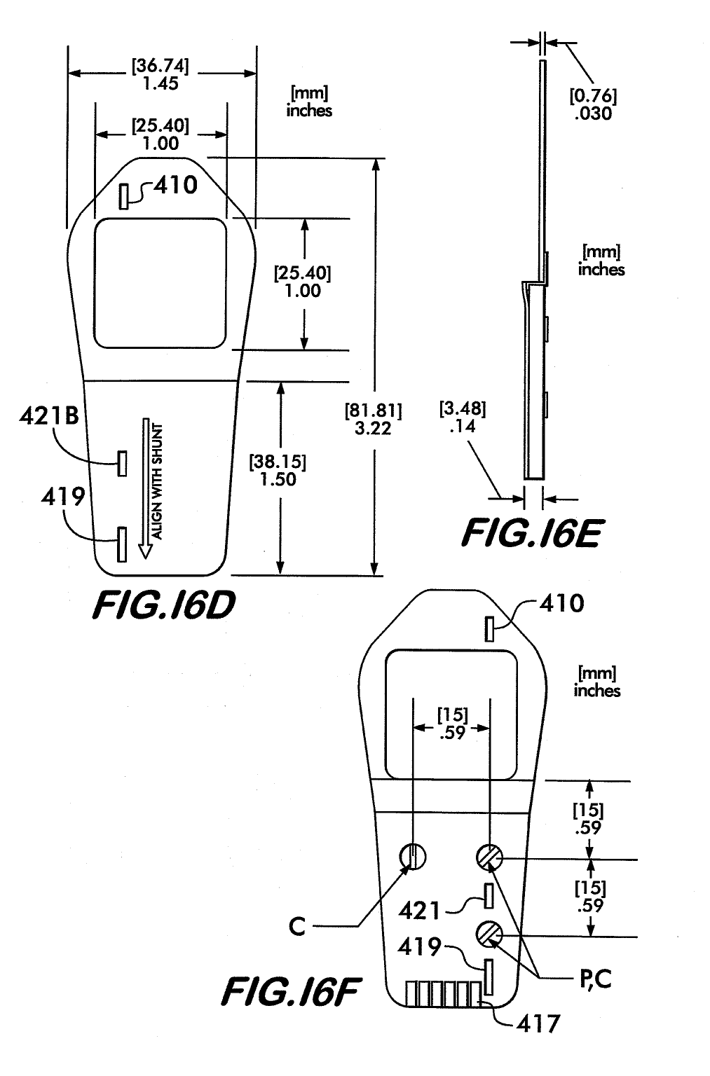

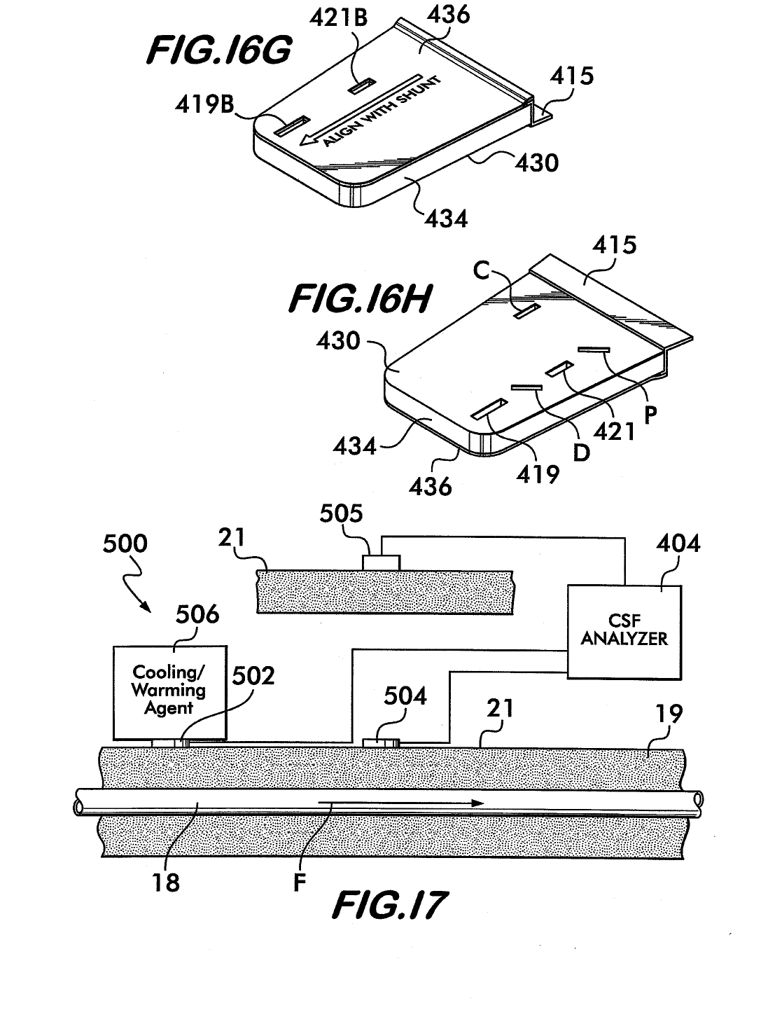

A further alternative embodiment 402B of the measurement pad is shown in FIGS. 16A- 16G which includes an insulation layer 415 (e.g., polymide thermal/moisture layer) that is provided between the window 442 in the first portion 408 (comprised of an absorbing material layer 438) and the thermistors P, C and D in the second portion 406. The polymide thermal/moisture layer 415 is adhesively secured to the lower portion 406 and to the border around the window 442. This layer and the adhesive provide the thermal isolation. Thus, it is also important to avoid the effects of putting a transverse air gap between the window 442 and the thermistors P, C and D. In addition, as can be seen most clearly in FIG. 16C, an insulation layer 434 (e.g., a poron foam layer) is provided on top of the substrate (e.g., polymide layer) containing the thermistors P, D and C to avoid accidental cooling of the thermistors directly from the window 408. A polymide layer 430 comprises the thermistors P, C and D in the second portion 406. This layer 430 further comprises the sensors' interface 417 (FIG. 16C) as well as apertures 419 and 421 that align with corresponding apertures 419A/421A and 419B/421B in the insulation layer 434 and a label 436, respectively. These apertures, like the aperture 410, provide additional means for properly aligning the measurement pad 402B over the shunt 18. The label 436 is secured to the insulation layer 434.

It should be understood that the dimensions provided in FIGS. 16A-16G are by way of example only and are not meant to limit the invention to those dimensions.

It should also be understood that although the thermistors P, C and D are shown as being coupled to the evaluation unit 404 via wires, it is within the broadest scope of the present invention to include a wireless interface between all of the thermistors P, C, and D and the evaluation unit 404. Thus, the type of interface between each of the sensors P, C, D (or any of the other configurations using a plurality of proximal or distal thermistors, etc.) and the evaluation unit 404 is not limited to what is shown but includes any type of wireless interface (RF, infrared, ultrasound, etc.).

It should be further noted that where the analyzer 404 operates in accordance with the CSF flow rate calculation system 95/shunt evaluation system circuitry 50 (see FIGS. 4-5), the number of input lines 54-60 can be adjusted accordingly to accommodate the particular number of sensors (e.g. thermistors) present (e.g., three inputs for P, C and D thermistors; or more for a plurality of P thermistors or D thermistors, etc.). The shunt evaluation system circuitry 50 processes the signals from these sensors to provide further electrical signals representative of the temperatures of the corresponding thermistors to permit the determination of the flow rate or flow status of the CSF through the shunt tubing 18 as previously described.

Referring now to FIG. 17, there is shown the CSF shunt evaluation system 500 of the present invention which reacts to changes of temperature on the skin surface. The CSF shunt evaluation system 500 is provided with two sensors 502 and 504 (e.g., thermistors such as MA100 Catheter Assembly or the GE NTC thermistor, as discussed previously with regard to the system 400) disposed at predetermined locations on the body of the patient for determining the existence of CSF flow F through the shunt tubing 18, and determining the flow status (i.e., flow or no flow) and the flow rate of the CSF flow F through the shunt tubing 18. The upstream sensor 502 measures the temperature directly from the temperature source 506, e.g., a cooling or warming agent while the downstream sensor 504 (or any other downstream sensor, not shown) measures the temperature over the shunt tubing 18 at some distance from the upstream sensor 502. The placement of the temperature source 506 directly upon the upstream sensor 502 yields the advantage of knowing the precise time of the application ofthe cooling/warming agent 506 and permits the measurement of the “input” temperature to the entire system 500 (i.e., shunt tubing 18, underlying tissue 19 and skin 21), which yields some additional possibilities of detection. For example, the input temperature profile T(t) can be detected downstream by other sensors and the time difference between the “input” and “downstream” profiles can be calculated which can lead to flow rate (or flow status) detection. Also, a control sensor 505 is used, along with the upstream and downstream sensors 502/504, to permit the calculation of error signals due to background effects such as body temperature and ambient temperature. The error signals within CSF shunt evaluation system 500 can be used to provide a more accurate determination of the CSF flow status or rate through the shunt tubing 18.

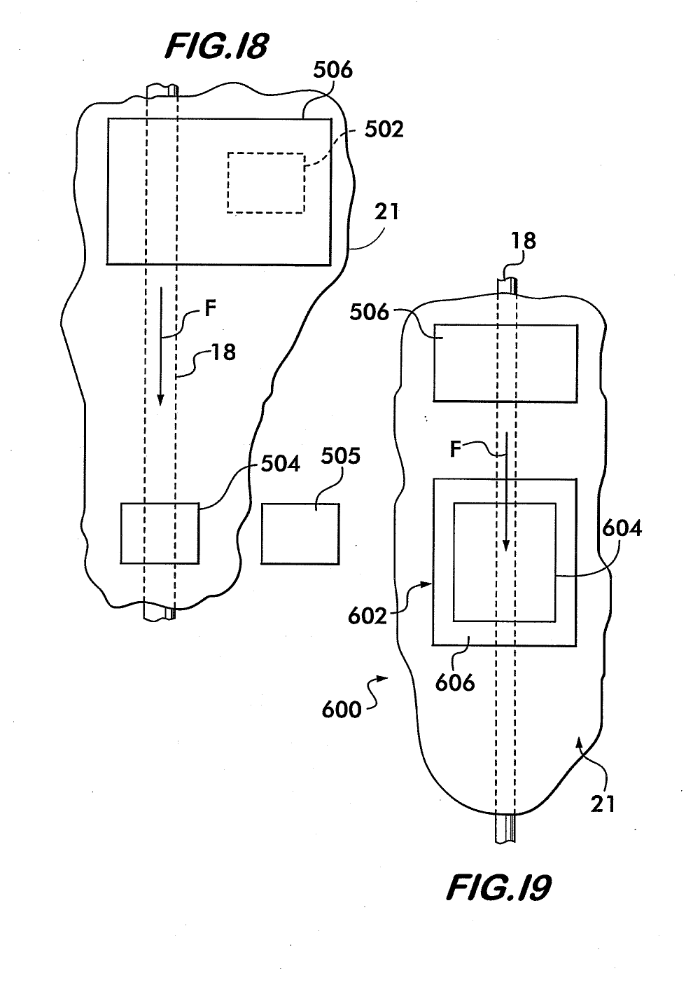

In particular, as shown in FIG. 18, the upstream sensor 502 is placed on the skin 21 but not over the shunt tubing 18. The reason for this is that with the temperature source 506 (e.g., cooling agent such as an ice pack) applied directly to the upstream sensor 502, the temperature source dominates the temperature detected by the sensor 502. Moreover, the temperature source 506 is large enough to apply such a temperature to the shunt tubing 18, the upstream sensor 502 and the surrounding skin 21, as shown in FIG. 18. The downstream sensor 504 is applied to the skin 21 at a position over the shunt tubing 18. The control temperature sensor 505 is applied to the skin 21 while being aligned with the downstream sensor 504, as can be seen in FIG. 18. As such, the application of the temperature source 506 not only conveys this forced temperature to the upstream sensor 502 and to the shunt tubing 18, but it also is applied to the skin 21 and it is this conduction through the skin 21 that is detected by the control sensor 505. The alignment assures that the downstream sensor 504 detects the temperature delta via the shunt tubing 18 while the temperature delta propagated via the skin 21 is detected by the control sensor 505. By way of example only, the distance between the edge of the temperature source 106 and the downstream sensor 504/control sensor 505 is approximately 15 mm, although this is provided by way of example and not limitation. The control sensor 505 thus provides electrical output signals representative of the detected temperature delta transmitted through the skin 21. The output signals from the control sensor 505 permits control readings to be performed by the CSF evaluation system 500 for error correction of the flow rate calculations that can be obtained using the sensors 502 and 504. Thus, the system 500 includes an evaluation unit 404 with which all of the sensors 502/505 are in communication. The evaluation unit 404 collects and processes the sensor data, as discussed earlier with regard to the CSF evaluation system 400.

In the method of the invention, the upstream sensor 502 is placed near the shunt tubing 18 (but not over it), for example, in the vicinity of an ear of the patient for providing an electrical output signal representative of the temperature of the CSF and upon which the temperature source 506 (e.g., ice pack) is positioned directly. The downstream sensor 504 is placed over the shunt tubing 18 in the vicinity of the clavicle of the patient for providing an electrical output signal representative of temperature of the CSF therebelow.

Preferably the sensors 502 and 504 can be disposed as close as possible to each other, as long as they are placed in an area where the shunt tubing 18 is substantially close to the surface of the body. The shunt tubing 18 is usually sufficiently close to the surface behind the pinna and on the neck. It is also close to the surface over the clavicle, which is often approximately fifteen centimeters from the pinna. Thus, in one preferred embodiment of the invention the spacing between the sensors 502/504 can be approximately fifteen centimeters or less. Furthermore, in one preferred embodiment the sensors 502/504 can be placed as close together as approximately three centimeters.

It should be understood that although the sensors 502/504 and control sensor 505 are shown as being coupled to the evaluation unit 404 via wires, it is within the broadest scope of the present invention to include a wireless interface between all of the sensors 502-505 and the evaluation unit 404. Thus, the type of interface between each of the sensors 502-505 and the evaluation unit 404 is not limited to what is shown but includes any type of wireless interface (RF, infrared, ultrasound, etc.).

Referring back to FIG. 4, the shunt evaluation system circuitry 50 can be used for receiving and processing the electrical output signals provided by the sensors 502/504 and the control sensor 505 of the CSF shunt evaluation system 500. It should be understood that with the reduction to three sensors (502, 504 and 505) in the present system 500, the input line 60 can be omitted, thus utilizing input lines 54-58 only. The shunt evaluation system circuitry 50 processes the signals from the sensors 502/504 and the control sensor 505 to provide further electrical signals representative of the temperatures of the sensors 502/504 and ofthe control sensor 505 to permit the determination of the flow rate or flow status of the CSF through the shunt tubing 18 as previously described.

The output signals of the sensors 502/504 and the control sensor 505 applied to the body of the patient are received at the input lines 54-58 of the evaluation system circuitry 50. In one preferred embodiment of the invention, the signals received on the input lines 54-58 can be sequentially switched onto a common input line 62 of a general purpose precision timer 68. Additionally, in an alternate embodiment of the invention, the signals on the input lines 54-58 can be applied to an analog-to-digital converter (not shown) to provide digital signals representative of the output of the sensors 502/504 and the control sensor 505 suitable for processing within the evaluation system circuitry 50.

The precision timer 68 of the evaluation system circuitry 50 that sequentially receives the signals from the sensors 502/504 and the control sensor 505 is adapted to operate as a relaxation oscillator circuit 70 having a varying output frequency related to a varying RC time constant. The precision timer 68 within the relaxation oscillator circuit 70 can be the well known ICM7555 or any other equivalent device.

The precision timer 68 is coupled to a capacitor 72 and to the common input line 62 of the three input lines 54-58. Each of the sensors 502/504 and the control sensor 505 coupled in sequence to the common input line 62 operates as a variable resistor whose resistance varies with a sensed temperature as previously described. The sequential coupling of the sensors 502/504 and the control sensor 505 to the capacitor 72 permits RC time constant within the relaxation oscillator circuit 70 to vary when the sensors 502/504 and the control sensor 505 sense different temperatures. Thus, the varying RC time constant results in varying frequencies of oscillation for the relaxation oscillator circuit 70 that correspond to the varying temperatures sensed by the sensors 502/504 and the control sensor 505.

When the relaxation circuit 70 of the shunt evaluation system circuitry 50 oscillates a battery 64 charges the capacitor 72 according to the resistance of the sensors 502/504 and the control sensor 505 coupled to the capacitor 72. This causes the voltage across the capacitor 72 to rise. When the voltage across the capacitor 72 rises to a predetermined level, the precision timer 62 triggers. The triggering of the precision timer 68 causes the capacitor 72 to discharge through the precision timer 62 by way of the line 74, thereby completing one cycle of the relaxation oscillator 70. The time period it takes for the capacitor 72 to charge to the predetermined voltage level and trigger is determined by the amount of charging current, and thus the amount of resistance, of the sensor 502/504 and the control sensor 505 coupled to the common input line 62. Thus, the oscillation frequency of the relaxation oscillator 70 is determined by the resistance, and thus the temperature, of the active sensors 502/504 and control sensor 505.

The use of the relaxation oscillator 70 for obtaining an electrical signal representative of the resistance of the sensors 502/504 and the control sensor 505 suitable for algorithmic processing is believed to be easier and less expensive than the use of an analog-to-digital converter for this purpose. Additionally, use of the relaxation oscillator 70 is believed to be more noise resistant than an analog-to-digital converter. Furthermore, the relaxation oscillator 70 uses less power than an analog-to-digital converter uses.

The frequency signal output of the precision timer 68 is applied to an input pin of a microprocessor 80 of the shunt evaluation system circuitry 50. The microprocessor 80 can be an AT90S2313 8-bit microcomputer, or any other microprocessor known to those skilled in the art. In addition to controlling the sequential switching of the sensors 502/504 and the control sensor 505 onto the common input line 62, the microprocessor 80 can operate as a frequency counter to determine a frequency value in accordance with the oscillation frequency of the relaxation oscillator 70. The frequency value determined by the microprocessor 80 is provided as an output of the shunt evaluation system circuitry 50 on an output bus 85. The output bus 85 can be coupled to a conventional RS-232 transceiver. In keeping with the system of the present invention, the output frequency value can also be provided on a parallel bus.

Referring back to FIG. 5, within the CSF flow rate/status calculation system 95 the computer 90 receives the frequency values determined by the shunt evaluation system circuitry 50 by way of the output bus 85. When the frequency values are received, the computer 90 performs calculations on them in order to determine the flow rate of the CSF through the shunt tubing 18 ofthe system 100 under the control of a stored program. Signals from the sensors 502 and 504 can be used by the computer 90 to calculate the flow rate/flow status through the shunt tubing 18 as previously described. For example, the flow rate calculation set forth above with respect to the CSF shunt evaluation system 500 can then be used to determine the CSF flow rate/status in accordance with the determined time difference 112. Signals from the control sensor 505 can be used to determine an error correction signal representative of background conditions for use in correcting the calculations performed on the signals from the sensors 502/504.

Referring back to FIG. 6, there is shown the response times of the sensors 502/504 within the CSF flow rate calculation system 95. The inflection point of the temperature inflection curve 104, representing the temperature of the upstream sensor 502, occurs first since the temperature source 106 is applied directly to the upstream sensor 502. The curve 104 inflection point occurs at time 108. At a time thereafter, varying according to the flow rate/status of the CSF, the inflection point of the curve 102 occurs. Curve 102 represents the temperature of the downstream sensor 504. The temperature infection curve 102 inflection point occurs at time 110. A skilled practitioner, preferably a neurosurgeon, determines the time difference 112 between the inflection points 108, 110.

In the error correction protocol, the skin temperature of the control sensor 505 is subtracted from the skin temperature at the location of upstream sensor 502 and also subtracted from the skin temperature at the location of the downstream sensor 504. These subtractions correct for global skin temperature changes such as changes due to environment and physiology, for example excitement, attention and pain, and provide error correction for adjusting the flow rate to provide a corrected CSF flow rate or status.

Using the correct (subtracted) temperature curve makes it possible in a realistic clinical situation to accurately detect inflection points as the cooled CSF passes under the thermistors. When the time of the inflection points 108, 110 and the time difference between the inflection points 108, 110 are determined the flow calculation can be performed in substantially the same manner as the flow calculations of the prior art.

For example, in one embodiment of the invention the software providing graphical representation 100 displays on the screen two temperature inflection curves 102, 104 one for the upstream (shunt temperature minus control temperature) pair of thermistors and one for the downstream (shunt temperature minus control temperature) pair. The operator can use a mouse to move two vertical bars to the inflection points 108, 110.

The software can provide a window showing the times corresponding to the inflection points 108, 110 selected and prompting the operator for the diameter of the tubing. Since only two diameters are in common clinical use, the window can allow a choice between these two in the preferred embodiment. The software then calculated the flow rate from the time difference and the diameter.

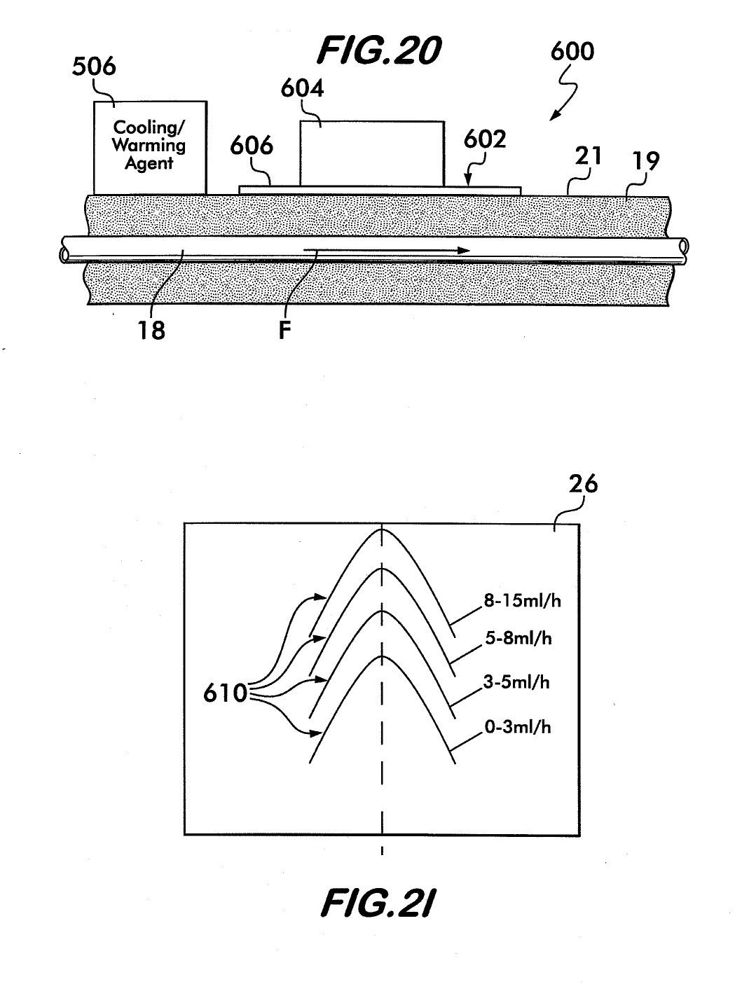

Referring now to FIG. 19, there is shown a device 600 that includes a liquid crystal material 602, e.g., sheet or film (as shown more clearly in FIG. 20) or spray, that is applied against the skin 21 of a subject in whom a subcutaneous tube 18 (e.g., a shunt tube) is disposed.

One exemplary manner of applying the temperature sensitive material is via a flexible liquid crystal sheet such as the Mylar® liquid crystal sheets/films sold by Anchor Optics (AX61161, AX72375, etc.), or by Educational Innovations (LC-3035A, LC-5A, etc.) or by LCR Hallcrest, etc. When the thermo-sensitive sheet 602 is applied to a surface, e.g., the skin 10 of the subject, the sheet 602 changes color corresponding to a temperature change. Therefore, using the method of the present invention 602, the liquid crystal sheet 602 is applied on the skin 21 directly over the location of a shunt tube. Next, a temperature source 506 (e.g., an ice pack, a Peltier junction/device, a heat source using solid state or other heaters, or any type of cooling/warming agent) is applied to the skin at an upstream location with respect to the liquid crystal sheet 602. The cold/hot input from the source 506 is conveyed to the flow F in the shunt and which then moves through the shunt tube 18. When the cold/hot input from the source 506 arrives at the liquid crystal sheet 602, the sheet 602 experiences the temperature change and correspondingly changes color. By conducting tests with various flow rates and applying a liquid crystal sheet over a subcutaneous test shunt tube when a temperature source is applied over the subcutaneous tube upstream of the liquid crystal sheet 602, a correlation of flow rates and color changes can be obtained. An example of such a correlation can be seen in FIG. 21 where the curved lines indicate color change profiles 610 that correspond to particular flow rates. Furthermore, in some cases, a flow status, (i.e., either flow is occurring or flow is blocked), rather than a flow rate can be determined from the liquid crystal sheet 602. As can appreciated by one skilled in the art, various other special grids may be used that relate color patterns (or other parameter patterns, e.g. light and dark reflections, etc.) on the liquid crystal sheet or other thermo sensitive material to a specific flow status or flow rate.

To facilitate such readings, the liquid crystal sheet 602 is configured in the device using a reading unit 604 (e.g., devices having picture analysis software, including color analysis, e.g., specialized digital cameras, including colorimeters that analyze colors; by way of example only, the DR/890 Colorimeter marketed by the Hach Company of Loveland, Colo., can be modified for use as the reading unit 604). Thus the top surface 606 (FIGS. 19-20) may comprise a grid, graduations, or other indicia, such as that shown in FIG. 21. Thus, when the device 600 is applied against the skin 21 under which the subcutaneous tube 18 is located, the liquid crystal sheet 602 comes into direct contact with the skin 21. The color profile is noted before the temperatures source is applied. Next, the temperature source 506 is applied to the skin 21, upstream of the device 600's location in which case the temperature input is then conveyed to the fluid in the subcutaneous tube 18. When the cooled (or heated) fluid reaches the portion of the tube 18 over which the sheet 602 is positioned, the temperature change causes the particular color profile to change and the user can use the reading unit 604 indicia to read off the flow rate therefrom. Alternatively, the grid can be imprinted directly on the sheet 602.

A further variation of the liquid crystal sheet 602 is that instead of its color or optical properties (e.g., polarization, attenuation, scattering, etc.) varying with temperature, it is possible that the electrical properties (e.g., resistivity, electrical permittivity, etc.) may vary with temperature. Moreover, the physical properties (elasticity, viscosity, etc.) of the liquid crystal sheet 602 may vary with temperature. It should be understood that where the electrical or physical properties vary according to temperature, the reading unit 604 may include means for interpreting such changes in the electrical/physical properties into flow status or flow rate, e.g., using a display with an alphanumeric readout.

It should be noted that an alternative to the liquid crystal sheet 602 is a temperature sensitive liquid that is sprayed-on the skin but which also changes color or other optical properties due to temperature changes. By way of example only, such a material is sold under the trademark Xposures® by The Alsa Corporation of Vernon, Calif. Alternatively, like the previously described variations of the liquid crystal sheet 602, the temperature sensitive liquid could also alter its electrical or physical properties in response to changes in temperature.

Another alternative is that the reading unit 604 is an active device, e.g., it is an optoelectronic or electronic means that analyze/interpret the color changes/patterns and provide a flow status (i.e., flow or no flow display) or a flow rate in alphanumeric form.

While the invention has been described in detail and with reference to specific examples thereof, it will be apparent to one skilled in the art that various changes and modifications can be made therein without departing from the spirit and scope thereof.

What is claimed is:

1. An apparatus for evaluating cerebrospinal fluid (CSF) flow rate or flow status in a CSF shunt applied to the body of a patient for transmitting said CSF between first and second locations of said body, said apparatus comprising:
a pad that is placed against the skin of a patient over the location of the CSF shunt, said pad comprising a pair of temperature sensors that are aligned in a first direction to form an upstream temperature sensor and a downstream temperature sensor with respect to the shunt, said pad further comprising a third temperature sensor that is not aligned in said first direction, each of said temperature sensors generating respective temperature data; and
a sensor processing device that is electrically coupled to said pad for receiving temperature data from each of said temperature sensors, said sensor processing device using said temperature data to determine a flow rate or flow status of said CSF through said shunt when a temperature source is applied to said pad.

2. The apparatus of claim 1 wherein each of said temperature sensors comprises a fast response thermistor having a time constant of less than 5 seconds.

3. The apparatus of claim 1 wherein said sensor processing device subtracts said temperature data of said third temperature sensor from said temperature data of said upstream sensor to form a first difference and subtracts said temperature data of said third temperature sensor from said temperature data of said downstream sensor to form a second difference.

4. The apparatus of claim 3 wherein each of said temperature sensors comprises a fast response thermistor having a time constant of less than 5 seconds.

5. The apparatus of claim 3 wherein a ratio of said first difference to said second difference is in the range of 1.5 to 4.0.

6. The apparatus of claim 3 wherein said sensor processing device determines that there is CSF flow if said first difference is ≧0.2° C. over a predetermined time period.

7. The apparatus of claim 3 wherein said sensor processing device determines that there is CSF flow if said second difference is ≧0.1° C. over a predetermined time period.

8. The apparatus of claim 3 wherein said pad comprises a temperature source placement location, said temperature source placement location being thermally insulated from said plurality of sensors.

9. The apparatus of claim 8 wherein said temperature source placement location has an edge that is positioned approximately 15 mm from said upstream sensor.

10. The apparatus of claim 9 wherein said downstream sensor is positioned approximately 15 mm from said upstream sensor.

11. The apparatus of claim 10 wherein said third temperature sensor is positioned approximately 15 mm from said upstream sensor.

12. The apparatus of claim 3 wherein said apparatus comprises an interlock that prevents said pad from being reconnected to said sensor processing device after said pad has been used.

13. The apparatus of claim 8 wherein said temperature source placement location comprises an area that distributes the temperature from said temperature source in a uniform or symmetric manner while a uniform pressure is applied.

14. The apparatus of claim 3 comprising indicia that permits an operator to precisely locate said pad on the skin over the shunt tubing.

15. The apparatus of claim 3 wherein said temperature source is a cooling means.

16. An apparatus for evaluating cerebrospinal fluid (CSF) flow rate or flow status in a CSF shunt applied to the body of a patient for transmitting said CSF between first and second locations of said body, said apparatus comprising:
a pad that is placed against the skin of a patient over the location of the CSF shunt, said pad comprising a pair of temperature sensors that are aligned in a first direction, one of said temperature sensors being positioned over the CSF shunt while said other temperature sensor is not positioned over the CSF shunt, each of said temperature sensors generating respective temperature data; and
a sensor processing device that is electrically coupled to said pad for receiving temperature data from each of said temperature sensors, said sensor processing device using said temperature data to determine a flow rate or flow status of said CSF through said shunt when a temperature source is applied to said pad.

17. The apparatus of claim 16 wherein each of said temperature sensors comprises a fast response thermistor having a time constant of less than 5 seconds.

18. The apparatus of claim 16 wherein said sensor processing device subtracts said temperature data of said other temperature sensor from said temperature data of said one temperature sensor to form a difference.

19. The apparatus of claim 18 wherein each of said temperature sensors comprises a fast response thermistor having a time constant of less than 5 seconds.

20. The apparatus of claim 18 wherein said sensor processing device determines that there is CSF flow if said difference is ≧0.2° C. over a predetermined time period.

21. The apparatus of claim 18 wherein said pad comprises a temperature source placement location, said temperature source placement location being thermally insulated from said plurality of sensors.

22. The apparatus of claim 21 wherein said temperature source placement location has an edge that is positioned approximately 15 mm from said one sensor.

23. The apparatus of claim 22 wherein said one temperature sensor is positioned approximately 15 mm from said other temperature sensor.

24. The apparatus of claim 18 wherein said apparatus comprises an interlock that prevents said pad from being reconnected to said sensor processing device after said pad has been used.

25. The apparatus of claim 19 wherein said temperature source placement location comprises an area that distributes the temperature from said temperature source in a uniform or symmetric manner while a uniform pressure is applied.

26. The apparatus of claim 18 comprising indicia that permits an operator to precisely locate said pad on the skin over the shunt tubing.

27. The apparatus of claim 18 wherein said temperature source is a cooling means.

28. The apparatus of claim 16 wherein said first direction comprises a direction that is perpendicular to the CSF shunt.

29. The apparatus of claim 28 further comprises a plurality of temperature sensors between said one temperature sensor and said other temperature sensor that are aligned along said first direction.

30. A method for evaluating cerebrospinal fluid (CSF) flow rate or flow status in a CSF shunt, said method comprising:
applying a pair of temperature sensors against the skin aligned with the CSF shunt to form an upstream temperature sensor and a downstream temperature sensor while simultaneously applying a third temperature sensor against the skin in the vicinity of the CSF shunt but not over the shunt;
applying a temperature source over the CSF shunt and upstream of said pair of temperature sensors for a predetermined period;
collecting temperature data after said predetermined period of time has elapsed;
subtracting temperature data of said third temperature sensor from each of the temperature data from said pair of temperature sensors to form first and second temperature differences respectively; and
determining a flow rate or flow status of the CSF through the shunt from said first and second temperature differences.

31. The method of claim 30 wherein said predetermined period is approximately 60 seconds.

32. The method of claim 30 wherein said step of applying a pair of temperature sensors comprises having the patient lie in a supine position for a second predetermined period of time before applying said temperature source.

33. The method of claim 30 wherein said step of applying a pair of temperature sensors comprises allowing said pair of temperature sensors and said third temperature sensor to remain against the skin for a third predetermined period of time.

34. The method of claim 30 wherein said step of applying a pair of temperature sensors comprises using three fast response thermistors each having a time constant of <5 seconds.

35. The method of claim 30 wherein said step of applying a temperature source comprises applying a cooling means.

36. The method of claim 30 wherein a ratio of said first difference to said second difference is in the range of 1.5 to 4.0.

37. The method of claim 30 wherein said step of determining a flow rate or flow status comprises determining that there is CSF flow if said first difference is ≧0.2° C. over a predetermined time period.

38. The method of claim 30 wherein said step of determining a flow rate or flow status comprises determining that there is CSF flow if said second difference is ≧0.1° C. over a predetermined time period.

39. The method of claim 30 wherein said step of applying a temperature source comprises applying said source no closer than approximately 15 mm to one of said pair of temperature sensors.

40. The method of claim 30 wherein said step of applying a pair of temperature sensors against the skin comprises positioning said pair of temperature sensors approximately 15 mm from each other.

41. The method of claim 30 wherein said third temperature sensor is positioned approximately 15 mm from said upstream temperature sensor.

42. The method of claim 30 wherein said step of collecting temperature data comprises preventing such data from being collected if said temperature sensors have been used previously.

43. The method of claim 30 wherein said step of applying a temperature source comprises distributing the temperature from said temperature source in a uniform or symmetric manner while applying a uniform pressure.

44. The method of claim 30 wherein said step of applying a pair of temperature sensors to the skin comprises fixing said pair of temperature sensors in relation to each other in a pad which includes indicia that can be referenced to marks previously made on the skin.

45. The method of claim 30 wherein said further comprising the step of applying at least a fourth temperature sensor are aligned with said downstream sensor in a direction that is perpendicular to the CSF shunt.

46. A method for evaluating cerebrospinal fluid (CSF) flow rate or flow status in a CSF shunt, said method comprising:
applying first and second temperature sensors against the skin wherein said first temperature sensor is positioned over the CSF shunt and the second temperature sensor is applied against the skin in the vicinity of the CSF shunt but not over the shunt;
applying a temperature source over the CSF shunt and upstream of said first temperature sensor for a predetermined period;
collecting temperature data after said predetermined period of time has elapsed;
subtracting temperature data of said second temperature sensor from the temperature data of said first temperature sensor to form a temperature difference; and
determining a flow rate or flow status of the CSF through the shunt from said temperature difference.

47. The method of claim 46 wherein said predetermined period is approximately 60 seconds.

48. The method of claim 46 wherein said step of applying first and second temperature sensors comprises having the patient lie in a supine position for a second predetermined period of time before applying said temperature source.

49. The method of claim 46 wherein said step of applying first and second temperature sensors comprises allowing temperature sensors to remain against the skin for a third predetermined period of time.

50. The method of claim 46 wherein said step of applying first and second temperature sensors comprises using fast response thermistors each having a time constant of <5 seconds.

51. The method of claim 46 wherein said step of applying a temperature source comprises applying a cooling means.

52. The method of claim 46 wherein said step of determining a flow rate or flow status comprises determining that there is CSF flow if said difference is ≧0.2° C. over a predetermined time period.

53. The method of claim 46 wherein said step of applying a temperature source comprises applying said source no closer than approximately 15 mm to said first temperature sensors.

54. The method of claim 46 wherein said step of applying said first and second temperature sensors against the skin comprises positioning said temperature sensors approximately 15 mm from each other.

55. The method of claim 46 wherein said step of collecting temperature data comprises preventing such data from being collected if said temperature sensors have been used previously.

56. The method of claim 46 wherein said step of applying a temperature source comprises distributing the temperature from said temperature source in a uniform or symmetric manner while applying a uniform pressure.

57. The method of claim 46 wherein said first and second temperature sensors are aligned in a direction that is perpendicular to the CSF shunt in a first direction, said method further comprising the step of applying at least a third temperature sensor between said first and second temperature sensors aligned in said first direction.

58. A method for evaluating cerebrospinal fluid (CSF) flow rate or flow status in a CSF shunt applied to the body of a patient for transmitting said CSF between first and second locations of said body, comprising:
applying a first temperature sensor at a first location external to said body in a vicinity of the CSF shunt and applying a second temperature sensor at a second location external to said body and under which the CSF shunt is located, said first location being upstream of said second location;
applying a control temperature sensor at a third location under which the CSF shunt is not located but which is aligned with said second temperature sensor, said control temperature sensor providing temperature correction signals representative of a temperature of said exterior of the body;
applying a temperature source directly to said first temperature sensor;
determining a flow rate or flow status of said CSF through said shunt to provide a determined CSF flow rate or flow status; and
adjusting said determined CSF flow rate in accordance with said temperature correction signals to provide a CSF flow rate corrected in accordance with said background temperature.

59. The method of claim 58 wherein said temperature sensors comprise thermistors.

60. The method of claim 58 wherein said temperature source comprising a cooling agent.

61. The method of claim 3 further comprising the step of measuring a temperature value of said CSF in accordance with said cooling.

62. The method of claim 61 further comprising the step of determining a time value in accordance with said temperature value.

63. The method of claim 62 further comprising step of determining said determined CSF flow rate in accordance with said time value.

64. The method of claim 63 further comprising the step of determining said determined CSF flow rate in accordance with a plurality of temperature values.

65. An apparatus for evaluating cerebrospinal fluid (CSF) flow rate or flow status in a CSF shunt applied to the body of a patient for transmitting said CSF between first and second locations of said body, said apparatus comprising:
a first temperature sensor, positioned at a first location external to the body and in the vicinity of the CSF shunt and providing first temperature outputs;
a second temperature sensor, positioned at a second location external to the body and under which the CSF shunt is located and providing second temperature outputs, said second location being downstream of said first location;
a control temperature sensor, positioned at a third location external to the body and aligned with said second temperature sensor for providing temperature correction signals representative of a temperature of the exterior of the body and forming third temperature outputs;
a sensor processing unit, in communication with said first and second temperature sensors and with said control temperature sensor, said sensor processing unit using said first through said third temperature outputs for determining a flow rate or flow status of said CSF through said shunt when a temperature source is applied directly to said first temperature sensor.

66. The apparatus of claim 65 wherein said third temperature outputs are used to correct said first and second temperature outputs in accordance with said background temperature correction signal to provide a CSF flow rate corrected in accordance with said background temperature.

67. The apparatus of claim 65 wherein said temperature sensors comprise thermistors.

68. The apparatus of claim 65 wherein said temperature source is a cooling agent.

69. A device for detecting or quantifying fluid flow in a subcutaneous tube of a subject, said device comprising a temperature sensitive material having properties that change with temperature, said temperature sensitive material being applied to the skin of the subject over the subcutaneous tube; and wherein a temperature change, applied to the skin at an upstream location of the subcutaneous tube, alters a property of said temperature sensitive material when it arrives at said material, said temperature sensitive material providing a correlation between the property change and flow status or flow rate.

70. The device of claim 69 wherein said temperature sensitive material is a liquid crystal sheet having an optical property that changes with temperature.

71. The device of claim 70 wherein said optical property is color.

72. The device of claim 71 wherein said flexible liquid crystal sheet includes indicia that correlates color change with flow status or flow rate.

73. The device of claim 71 further comprising a color detector that interprets the color change with a flow status or flow rate and displays such information alphanumerically.

74. The device of claim 70 wherein said optical property is polarization.

75. The device of claim 70 wherein said optical property is attenuation.

76. The device of claim 70 wherein said optical property is scattering.

77. The device of claim 69 wherein said temperature sensitive sheet is a liquid crystal sheet having an electrical property that changes with temperature.

78. The device of claim 77 wherein said electrical property is resistivity.

79. The device of claim 77 wherein said electrical property is electrical permittivity.

80. The device of claim 69 wherein said temperature sensitive sheet is a liquid crystal sheet having a physical property that changes with temperature.

81. The device of claim 80 wherein said physical property is elasticity.

82. The device of claim 80 wherein said physical property is viscosity.

83. The device of claim 69 wherein said temperature sensitive material is a liquid crystal spray that is applied to the skin of the subject over the subcutaneous tube.

84. The device of claim 83 wherein said liquid crystal spray has an optical property that changes with temperature.

85. The device of claim 84 wherein said optical property is color.

86. The device of claim 84 wherein said optical property is polarization.

87. The device of claim 84 wherein said optical property is attenuation.

88. The device of claim 84 wherein said optical property is scattering.

89. The device of claim 83 wherein said liquid crystal spray has an electrical property that changes with temperature.

90. The device of claim 89 wherein said electrical property is resistivity.

91. The device of claim 89 wherein said electrical property is electrical permittivity.

92. The device of claim 83 wherein said liquid crystal spray has a physical property that changes with temperature.

93. The device of claim 96 wherein said physical property is elasticity.

94. The device of claim 90 wherein said physical property is viscosity.

95. A method for detecting or quantifying fluid flow in a subcutaneous tube of a subject, said method comprising:
applying a temperature sensitive material having properties that change with temperature, to the skin of the subject over the subcutaneous tube;
applying a temperature source to the skin of the subject at an upstream location with respect to said temperature sensitive material; and
correlating changes in properties of said temperature sensitive material with different flow rates for indicating flow status or flow rate.

96. The method of claim 95 wherein said temperature sensitive material is a liquid crystal sheet having an optical property that changes with temperature.

97. The method of claim 95 wherein said step of correlating changes comprises including indicia with said liquid crystal sheet that correlates temperature profiles with different flow rates for indicating flow status or flow rate.

98. The method of claim 96 wherein said optical property is color.

99. The method of claim 96 wherein said optical property is polarization.

100. The method of claim 96 wherein said optical property is attenuation.

101. The method of claim 96 wherein said optical property is scattering.

102. The method of claim 96 wherein said temperature sensitive material is a liquid crystal sheet having an electrical property that changes with temperature.

103. The method of claim 102 wherein said electrical property is resistivity.

104. The method of claim 103 wherein said electrical property is electrical permittivity.

105. The method of claim 95 wherein said temperature sensitive material is a liquid crystal sheet having a physical property that changes with temperature.

106. The method of claim 104 wherein said physical property is elasticity.

107. The method of claim 104 wherein said physical property is viscosity.

108. The method of claim 95 wherein said temperature sensitive material is a liquid crystal spray having an optical property that changes with temperature.
